(19) United States
(12) Patent Application Publication
(10) Pub. No.: US 2012/0282184 A1
(43) Pub. Date: Nov. 8, 2012

(54) TREATMENT OF OBESITY

(75) Inventors: Herman Waldmann, Oxford (GB); Kathleen Nolan, Marston (GB); Lynn Poulton, Beecroft (AU)

(73) Assignee: ISIS INNOVATION LTD, Oxfordshire (GB)

(21) Appl. No.: 13/504,647

(22) PCT Filed: Oct. 29, 2010
(86) PCT No.: PCT/GB2010/051819

§ 371 Date: Jul. 30, 2012

(30) Foreign Application Priority Data
Oct. 30, 2009 (GB) 0919054.7

(51) Int. Cl. A61K 39/395; A61K 49/00; A61P 3/04; A61P 3/10; A61P 3/00; A61P 3/06; C07K 16/28; G01N 33/573; C12Q 1/32; C12Q 1/52; G01N 33/68; G01N 27/72; G01N 33/66; G01N 33/92; G01N 33/64; G01N 33/74; A61K 38/17; A61K 38/28

(52) U.S. Cl. 424/9.2; 424/173.1; 424/144.1; 424/133.1; 514/6.5; 514/4.8; 530/389.6; 435/7.4; 436/501; 435/26; 435/16; 436/86; 436/95; 436/71; 436/128; 436/89; 514/1.1; 514/6.9

(57) ABSTRACT

A method for the prevention or treatment of a condition selected from type 2 diabetes, obesity and metabolic syndrome comprising activating a GITRL pathway. Methods for screening for substances to treat these conditions, diagnosing or predicting these conditions and selecting and monitoring therapies.

### FIELD OF THE INVENTION

The present invention relates to methods for preventing and treating conditions such as type 2 diabetes, obesity and metabolic syndrome by activating a GITRL pathway. In one embodiment, the invention relates to the use of an anti-GITR antibody for the treatment of these conditions. The invention also relates to a screening method for identifying substances useful for the treatment of these conditions.

### BACKGROUND OF THE INVENTION

Obesity is defined as too much body fat. Obesity is recognized as a major risk factor for coronary heart disease, which can lead to heart attack. It has also been associated with many chronic conditions, including: hypertension, coronary heart disease, type 2 diabetes, osteoarthritis and sleep apnea. Obesity is recognized to raise blood cholesterol and triglyceride levels, lower HDL cholesterol (thereby increase risk of heart disease and stroke) and raise blood pressure. It is also a major cause of gallstones and can worsen degenerative joint disease.

Obesity rates in the Western world have increased dramatically in the past 25 years. However, it has been clearly demonstrated that weight loss alleviates many of these symptoms and reduces the severity of many of the chronic conditions listed above. Further, weight loss prevents future illnesses by controlling underlying risk factors. Reducing body weight has been shown to protect against cardiovascular disease by lowering blood pressure, total cholesterol, LDL cholesterol, and triglycerides.

Diabetes mellitus is a major cause of morbidity and mortality. Chronically elevated blood glucose leads to debilitating complications including nephropathy, often necessitating dialysis or renal transplant; peripheral neuropathy; retinopathy leading to blindness; ulceration of the legs and feet, leading to amputation; fatty liver disease, which may progress to cirrhosis; and susceptibility to coronary artery disease and myocardial infarction.

Type 2, or noninsulin-dependent diabetes mellitus (NIDDM) typically develops in adulthood. NIDDM is associated with resistance of glucose-utilizing tissues like adipose tissue, muscle, and liver, to the actions of insulin. Initially, the pancreatic islet beta cells compensate by secreting excess insulin, but eventual islet failure results in decompensation and chronic hyperglycemia. There are several classes of drugs that are useful for treatment of NIDDM: 1) substances which directly stimulate insulin release, but carry the risk of hypoglycemia; 2) substances which potentiate glucose-induced insulin secretion, and must be taken before each meal; 3) biguanides, including metformin, which attenuate hepatic gluconeogenesis (which is paradoxically elevated in diabetes); 4) substances which improve peripheral responsiveness to insulin, for example the thiazolidinedione derivatives rosiglitazone and pioglitazone, but which have side effects like weight gain, oedema, and occasional liver toxicity; 5) insulin injections, which are often necessary in the later stages of NIDDM when the islets have failed under chronic hyperstimulation.

Metabolic Syndrome is a cluster of metabolic risk factors, including elevated blood glucose, glucose intolerance, insulin resistance, elevated triglycerides, elevated LDL-cholesterol, low high-density lipoprotein (HDL) cholesterol, elevated blood pressure, abdominal obesity, pro-inflammatory states, and pro-thrombotic states. Individuals with metabolic syndrome are at increased risk of cardiovascular disease and coronary heart disease and other diseases related to plaquing of the artery walls and type 2 diabetes. Some of the risk factors of cardiovascular disease and coronary heart disease include impaired fasting glucose, elevated blood lipids (total cholesterol, LDL-cholesterol, triglycerides), low HDL-cholesterol, obesity, type 2 diabetes, elevated blood pressure, insulin resistance, and proinflammatory and pro-thrombotic factors.

Glucocorticoid-induced tumor necrosis factor (TNF) receptor family-related gene (GITR), also known as TNF receptor superfamily member 18 (TNFRSF18), TEASR, and 312C2, is a type I transmembrane protein with homology to TNF receptor family members. GITR is a 241 amino acid type I transmembrane protein characterized by three cysteine pseudorepeats in the extracellular domain. The nucleic acid and amino acid sequences of human GITR (hGITR), of which there are three splice variants, are known and can be found in, for example GenBank Accession Nos. gi:40354198, gi:23238190, gi:23238193, and gi:23238196. GITR is expressed at low levels on resting CD4+ and CD8+ T cells and up-regulated following T-cell activation. Ligation of GITR provides a costimulatory signal that enhances both CD4+ and CD8+ T-cell proliferation and effector functions. In addition, GITR is expressed constitutively at high levels on regulatory T cells.

The mouse and human GITR-ligand (GITRL) genes are described in the NCBI database Entrez Gene at GeneID: 240873 and GeneID: 8995 respectively. It has been shown that the mouse ligand (GITRL) is upregulated on antigen presenting cells by inflammatory stimuli. Further, it was shown that engagement of GITR by GITRL leads to both dampening of the suppressive effects of regulatory T cells and to co-activation of effector T cells (Tone et al. 2003. Proc Natl Acad Sci U S A 100: 15059-64; Suvas et al. 2005. J Virol 79: 11935-42; Hisaeda et al. 2005. Eur J Immunol 35: 3516-24; Tuyaerts et al. 2007. J Leukoc Biol 82: 93-105). Mouse GITRL is located on distal chromosome 1 (location 1H2.1; 84.95cM), immediately adjacent to OX40L (TNFSF4), and close to FasL (TNFSF6). The region on distal chromosome 1 has been implicated to multiple metabolic traits (Chen et al. 2008. Nature 452: 429-35).

US 2009/0136494 suggests the use of a GITR binding molecule in a combination therapy for inhibiting tumor cell growth and reducing tumor size, with one or more additional agents, such as a chemotherapeutic agent. It also describes a humanized anti-GITR antibody.

Despite the existence of various drugs and therapies, obesity, diabetes and metabolic syndrome remain a major and growing public health problem. There is a need for new active therapeutic agents which are effective in preventing and treating these conditions.

### SUMMARY OF THE INVENTION

According to a first aspect the invention provides a method for the prevention or treatment of a condition selected from type 2 diabetes, obesity and metabolic syndrome comprising activating a GITRL pathway. Advantageously, this method provides a surprisingly effective way to prevent, control or treat these conditions.

In one embodiment, the method comprises administering a GITR-ligand or an analogue thereof or an agonist of a GITRL-associated receptor. The GITR-ligand or analogue thereof or the agonist of a GITRL-associated receptor may comprise a GITR-binding molecule or an antigen-binding fragment thereof. The GITR-ligand analogue or the agonist of a GITRL-associated receptor or the GITR-binding molecule or antigen-binding fragment may be an antibody or antibody fragment thereof. Preferably, the antibody or antibody fragment is a monoclonal antibody. Preferably, the antibody or antibody fragment is a chimeric antibody or fragment thereof or a humanized antibody or fragment thereof. Preferably, the antibody is an anti-GITR antibody. In another embodiment the GITR-ligand analogue or the agonist of a GITRL-associated receptor may comprise a small drug analogue or agonist.

The invention also provides a method for lowering cholesterol in a mammal which comprises activating a GITRL pathway. The method may comprise any of the features in the preceding paragraph or described herein in more detail below.

According to another aspect the invention provides a GITR-ligand or an analogue thereof or an agonist of a GITRL-associated receptor for the prevention or treatment of a condition selected from type 2 diabetes, obesity and metabolic syndrome or for use in lowering cholesterol in a mammal. The invention also provides a pharmaceutical composition comprising a GITR-ligand or an analogue thereof or an agonist of a GITRL-associated receptor for use in the prevention or treatment of a condition selected from type 2 diabetes, obesity and metabolic syndrome or for use in lowering cholesterol in a mammal. The invention further provides the use of a GITR-ligand or an analogue thereof or an agonist of a GITRL-associated receptor for the manufacture of a medicament for the prevention or treatment of a condition selected from type 2 diabetes, obesity and metabolic syndrome or for use in lowering cholesterol in a mammal. Preferred features of these embodiments are as discussed above and throughout the specification.

According to another aspect the invention provides a method for screening for a substance, or a salt or a solvate thereof, to be used in the prevention or treatment of a condition selected from type 2 diabetes, obesity and metabolic syndrome, or for use in lowering cholesterol in a mammal which comprises the following steps:

(i) measuring the severity of a symptom or sign of the condition in a non-human animal in which the GITRL gene is disrupted;

(ii) administering a test substance to said non-human animal; and

(iii) measuring the severity of a symptom or sign of the condition of said non-human animal after administration of the test substance.

(iv) comparing the severity of the symptom or sign of the condition before administration of the test substance with the severity of the symptom or sign of the condition after administration of the test substance, wherein a decrease or lessening of the symptom or sign indicates that the substance administered may be useful in the prevention or treatment of the condition.

The symptom or sign to be measured may be one or more of weight gain, fatty liver, levels of one or more of ALT, AST, lactate dehydrogenase, cholesterol, glucose, insulin, free fatty acids (FFAs), ketone bodies (e.g. BHBA) or interleukin-6 in the blood. Additional signs to be measured may include the levels of one or more of triglycerides, essential fatty acids (e.g. eicosapentaenoate, docosapentaenoate, dihomo-linolenate and docosahexaenoate), arginine, proline, ornithine, trans-4-hydroxyproline, corticosterone or arachnidonate in the blood. Combining information from two or more of these symptoms or signs may be confirmative.

According to yet another aspect the invention provides a method for diagnosing or predicting the risk of developing a condition selected from type 2 diabetes, obesity and metabolic syndrome in a subject, which comprises measuring the concentration of one or more biomarkers in the blood of or in a plasma sample from the subject, wherein the one or more biomarkers are selected from ALT, AST, lactate dehydrogenase, cholesterol, glucose, insulin, free fatty acids (FFAs), ketone bodies (e.g. BHBA), interleukin-6, triglycerides, essential fatty acids (e.g. eicosapentaenoate, docosapentaenoate, dihomo-linolenate and docosahexaenoate), arginine, proline, ornithine, trans-4-hydroxyproline, corticosterone, arachnidonate or citrulline. The concentration of the biomarker may be compared to the concentration of the biomarker in a healthy subject, wherein a change in the level of the biomarker is indicative of one or more of the conditions. Additionally or alternatively the concentration of the biomarker may monitored in the subject over a period of time, wherein a change in the level of the biomarker over time is indicative of one or more of the conditions. Combining information from two or more of these symptoms or signs may be confirmative.

The invention also provides a method for monitoring the effectiveness of a treatment of a condition selected from type 2 diabetes, obesity and metabolic syndrome comprising measuring the concentration of one or more biomarkers in the blood of or in a plasma sample from the subject, wherein the one or more biomarkers are selected from ALT, AST, lactate dehydrogenase, cholesterol, glucose, insulin, free fatty acids (FFAs), ketone bodies (e.g. BHBA), interleukin-6, triglycerides, essential fatty acids (e.g. eicosapentaenoate, docosapentaenoate, dihomo-linolenate and docosahexaenoate), arginine, proline, ornithine, trans-4-hydroxyproline, corticosterone, arachnidonate or citrulline. Combining information from two or more of these symptoms or signs may be confirmative.

The invention also provides a method for selecting an appropriate therapy for treating a condition selected from type 2 diabetes, obesity and metabolic syndrome in a patient comprising measuring the concentration of one or more biomarkers in the blood of or in a plasma sample from the subject, wherein the one or more biomarkers are selected from ALT, AST, lactate dehydrogenase, cholesterol, glucose, insulin, free fatty acids (FFAs), ketone bodies (e.g. BHBA), interleukin-6, triglycerides, essential fatty acids (e.g. eicosapentaenoate, docosapentaenoate, dihomo-linolenate and docosahexaenoate), arginine, proline, ornithine, trans-4-hydroxyproline, corticosterone, arachnidonate or citrulline. Combining information from two or more of these symptoms or signs may be confirmative.

### BRIEF DESCRIPTION OF THE DRAWINGS

FIG. 1 shows schematic representations of the GITRL gene locus in wild type mice and in the GITRL−/− knockout mouse. Shaded areas represent potential exons of the mouse GITRL gene. Horizontal bars represent the extent of the arms of the targeting construct used to replace exon 2 and the coding region of exon 3 of the wildtype gene with a neomycin cassette.

FIG. 2 shows growth curves of female WT C57B1/6J (n=4) and homozygous 6 generation backcrossed Gitrl−/− (n=4) mice.

FIG. 3 shows oil red O staining of liver sections from 6 month old C57B1/6J WT (left) and 6 generation backcrossed Gitrl−/− (right) mice.

FIG. 4 shows hematoxylin and eosin staining of livers of 2 control (left panels) and 3 Gitrl−/− mice (9th generation backcross) (right panels).

FIG. 5 shows serum IL-6 at 3 and 6 months in female C57B1/6J WT and 6 generation backcrossed Gitrl+/− and Gitrl−/− mice.

FIG. 6 shows serum insulin levels in 6 month old male C57B1/6J WT and 6 generation backcrossed Gitrl+/− and Gitrl−/− mice.

FIG. 7 shows SEQ ID NO:1, the Human GITRL Protein Sequence: Accession number NP_{—}005083.2.

FIG. 8 shows SEQ ID NO:2, the Human GITRL cDNA sequence: Accession number NM_{—}005092. Coding sequence is underlined. Exons are indicated as alternating non-bold and bold text, i.e. exon1-exon2-exon3.

FIG. 9 shows SEQ ID NO:3, the Mouse GITRL Protein Sequence: Accession number NP_{—}899247.

FIG. 10 shows SEQ ID NO:4, the Mouse GITRL cDNA Sequence: Accession number NM_{—}183391, variant 1 (3 exons). Coding sequences are underlined. Exons are indicated as alternating non-bold and bold text, i.e. exon1-exon2-exon3.

FIG. 11 shows SEQ ID NO:5, the Mouse GITRL cDNA Sequence: Accession number AJ577580, variant 2 (4 exons). Coding sequences are underlined. Exons are indicated as alternating non-bold and bold text, i.e. exon1-exon2-exon3-exon4.

FIG. 12 shows the weight of male control and knockout mice over time. E denotes experimental (i.e. GITRL−/− knockout) mice and C denotes control (i.e. wild type C57BL/6J) mice.

FIG. 13 shows the weight of female control and knockout mice over time. E denotes experimental (i.e. GITRL−/− knockout) mice and C denotes control (i.e. wild type C57BL/6J) mice.

FIG. 14 shows the weight difference between the female experimental and control groups is statistically significant from 100 days. 2-way ANOVA, with Bonferroni post test, used to determine if the weight difference is significant at a given time point. P value: ***<0.001; **0.001-0.01; *0.01-0.05; ns>0.05.

FIG. 15a shows the serum cholesterol levels of female knockout (diamond) and control (square) mice over time. FIG. 15b shows the serum cholesterol levels of male knockout (diamond) and control (square) mice over time.

FIG. 16a shows the serum glucose levels of female knockout (diamond) and control (square) mice over time. FIG. 16b shows the serum glucose levels of male knockout (diamond) and control (square) mice over time.

FIG. 17a shows the serum triglyceride levels of female knockout (diamond) and control (square) mice over time. FIG. 17b shows the serum triglyceride levels of male knockout (diamond) and control (square) mice over time.

FIG. 18a shows the serum insulin levels of female knockout (diamond) and control (square) mice over time. FIG. 18b shows the serum insulin levels of male knockout (diamond) and control (square) mice over time.

FIG. 19 shows the metabolomic data obtained for cholesterol in control and knockout mice over time. GLKO denotes GITRL−/− knockout mice and WT denotes control (i.e. wild type C57BL/6J) mice.

FIG. 20 shows the metabolomic data obtained for essential fatty acids in control and knockout mice over time. GLKO denotes GITRL−/− knockout mice and WT denotes control (i.e. wild type C57BL/6J) mice.

FIG. 21 shows the metabolomic data obtained for 3-hydroxybutyrate (BHBA) in control and knockout mice over time. GLKO denotes GITRL−/− knockout mice and WT denotes control (i.e. wild type C57BL/6J) mice.

FIG. 22 shows the different metabolic pathways for breaking down arginine (A) and metabolomic data obtained for arginine (B), citrulline (C), ornithine (D), proline (E), trans-4-hydroxyproline (F), urea (G), creatine (H), arachidonate (I) and corticosterone (J) in control and knockout mice over time. GLKO denotes GITRL−/− knockout mice and WT denotes control (i.e. wild type C57BL/6J) mice.

FIG. 23 shows the metabolomic data obtained for glucose in control and knockout mice over time. GLKO denotes GITRL−/− knockout mice and WT denotes control (i.e. wild type C57BL/6J) mice.

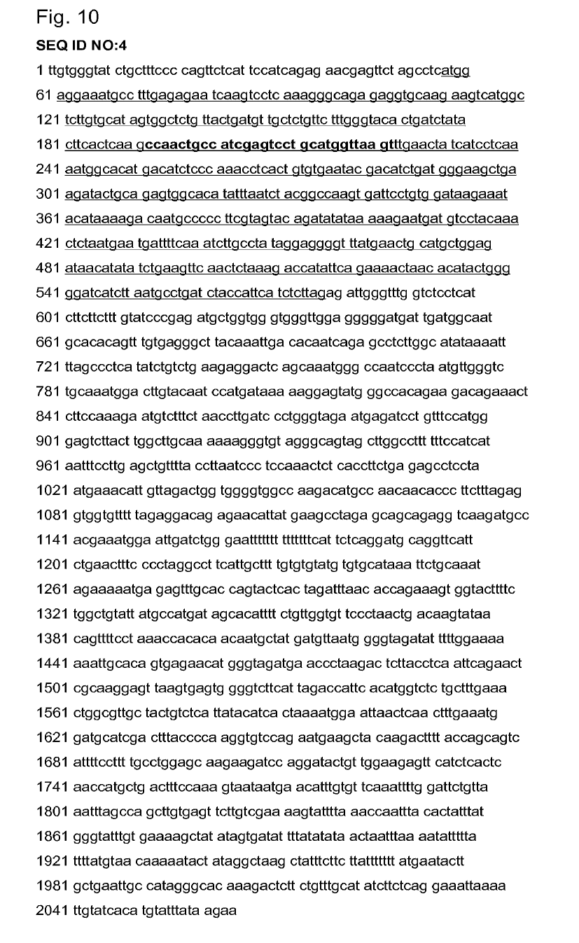

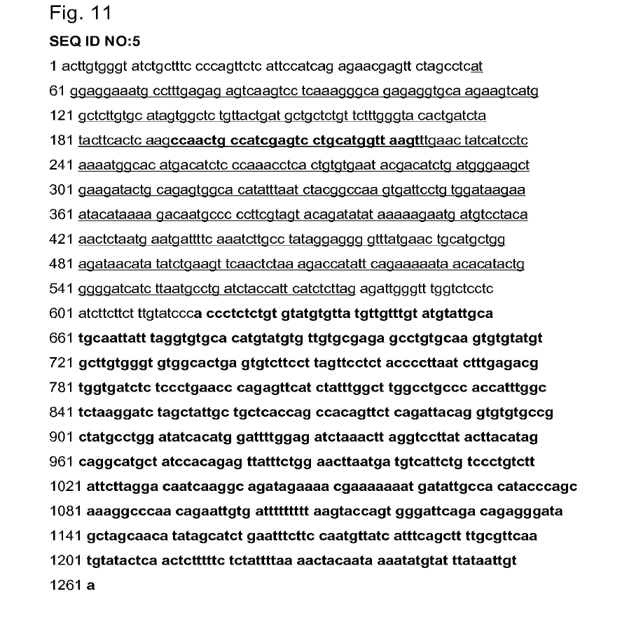

The 2 murine GITRL cDNA sequence accession numbers shown in FIGS. 10 and 11 represent alternatively spliced versions of the gene. Variant 1, NM_{—}183391, is encoded by 3 exons. The second variant, AJ577580, reported by Tone et al, PNAS 2003, 100:15059-, occurs by alternative splicing of a 4th exon replacing the 3′ untranslated region. The protein coding sequence is identical for both variants (FIG. 9, SEQ ID NO:3, NP_{—}899247, above).

### DETAILED DESCRIPTION OF THE INVENTION

The invention relates to a novel method for the prevention or treatment of conditions such as type 2 diabetes, obesity and metabolic syndrome by activating a GITRL pathway. The invention also relates to a novel method for lowering cholesterol in a mammal and for lowering the levels of other substances, such as inflammatory markers, which may predispose the mammal to harmful diseases, such as obesity, metabolic syndrome, type 2 diabetes, cardiovascular disease, atherosclerosis, myocardial infarction, stroke and peripheral vascular disease. It is shown herein that the GITRL plays an unexpected role as a physiological regulator of metabolism. More specifically, the knock-out of the GITRL gene confers on mice of the C57/B6 strain a propensity for weight gain (obesity), development of fatty liver, and increased levels of ALT, AST, lactate dehydrogenase, cholesterol, glucose, insulin, free fatty acids (FFAs), ketone bodies (e.g. BHBA) and interleukin-6 in the blood. Accordingly, it is believed that the GITR-ligand acts to normally prevent obesity and the development of metabolic syndrome and type 2 diabetes.

The conditions that may be prevented, controlled or treated in accordance with the invention include obesity, metabolic syndrome and type 2 diabetes. In general, the terms ‘prevent’, ‘control’ and ‘treat’ encompass the prevention of the development of a disease or a symptom from a patient who may have a predisposition of the disease or the symptom but has yet been diagnosed to have the disease or the symptom; the inhibition of the symptoms of a disease, namely, inhibition or retardation of the progression thereof; and the alleviation of the symptoms of a disease, namely, regression of the disease or the symptoms, or inversion of the progression of the symptoms.

All types of obesity may be controlled or treated in accordance with the invention, including endogenous obesity, exogenous obesity, hyperinsulinar obesity, hyperplastic-hypertrophic obesity, hypertrophic obesity, hypothyroid obesity and morbid obesity. However, inflammation-mediated obesity may be treated particularly effectively in accordance with the invention. By ‘prevent’ or ‘control’ or ‘treat’ it is meant that body weight gain, specifically body fat gain, is slowed down, stopped or reversed, resulting in a maintenance or decrease in body weight. A decrease in weight or body fat may protect against cardiovascular disease by lowering blood pressure, total cholesterol, LDL cholesterol and triglycerides, and may alleviate symptoms associated with chronic conditions such as hypertension, coronary heart disease, type 2 diabetes, osteoarthritis, sleep apnea and degenerative joint disease.

Metabolic syndrome is a cluster of metabolic risk factors. By ‘control’ or ‘treat’ it is meant that the symptoms of the metabolic syndrome shown in an individual are reduced in severity and/or in number. Such symptoms may include elevated blood glucose, glucose intolerance, insulin resistance, elevated triglycerides, elevated LDL-cholesterol, low high-density lipoprotein (HDL) cholesterol, elevated blood pressure, abdominal obesity, pro-inflammatory states, and pro-thrombotic states. By ‘prevent’ or ‘control’ or ‘treat’ it is additionally or alternatively meant that the risk of developing associated diseases is reduced and/or the onset of such diseases is delayed. Such associated diseases include cardiovascular disease, coronary heart disease and other diseases related to plaquing of the artery walls and type 2 diabetes.

Type 2, or noninsulin-dependent diabetes mellitus (NIDDM), is associated with resistance of glucose-utilizing tissues like adipose tissue, muscle, and liver, to the actions of insulin. Chronically elevated blood glucose associated with NIDDM can lead to debilitating complications including nephropathy, often necessitating dialysis or renal transplant; peripheral neuropathy; retinopathy leading to blindness; ulceration of the legs and feet, leading to amputation; fatty liver disease, which may progress to cirrhosis; and susceptibility to coronary artery disease and myocardial infarction. By ‘prevent’ it is meant that the risk of developing of diabetes is reduced or the onset of the disease is delayed. By ‘control’ or ‘treat’ it is meant that the risk of developing associated complications is reduced and/or the onset of such complications is delayed.

High levels of cholesterol are strongly associated with cardiovascular disease because these promote atherosclerosis. This disease process can lead to myocardial infarction (heart attack), stroke, and peripheral vascular disease. Lowering cholesterol levels in a mammal is therefore seen as important in preventing the development of these diseases. Similarly, lowering the levels of other substances, such as inflammatory markers (e.g. IL-6, products of arginase activity and citrullinated proteins), which may predispose the mammal to harmful diseases, such as obesity, metabolic syndrome, type 2 diabetes and cardiovascular disease, is also important seen as important in preventing the development of these diseases.

### Glucocorticoid-Induced Tumor Necrosis Factor Receptor Ligand (GITRL)

The present invention provides for activation of a GITRL pathway in order to prevent, treat or control a condition selected from type 2 diabetes, obesity and metabolic syndrome. Accordingly, in one aspect, the present invention provides a GITR-ligand or an analogue thereof or an agonist of a GITRL-associated receptor for the prevention or treatment of a condition selected from type 2 diabetes, obesity and metabolic syndrome. The invention also provides a pharmaceutical composition comprising a GITR-ligand or an analogue thereof or an agonist of a GITRL-associated receptor for use in the prevention or treatment of a condition selected from type 2 diabetes, obesity and metabolic syndrome. The invention further provides the use of a GITR-ligand or an analogue thereof or an agonist of a GITRL-associated receptor for the manufacture of a medicament for the prevention or treatment of a condition selected from type 2 diabetes, obesity and metabolic syndrome. Preferred features of these embodiments are as discussed above and below.

By “activating a GITRL pathway” it is intended that one or more substances are administered to an individual which can provide for GITRL-mediated signalling. GITRL-mediated signalling may be demonstrated, for example, by increased effector T cell response and/or increased humoral immunity, such as, for example, for those binding molecules described in US20070098719, US20050014224, and WO05007190. As shown in the examples, in an animal such GITRL-mediated signalling may result in a reduction in weight gain, fatty liver, or levels of ALT, AST, lactate dehydrogenase, cholesterol, glucose, insulin or interleukin-6 in the blood. Such signals may result from the interaction of GITRL with a GITRL-associated receptor, such as GITR, or may result from other interactions involving GITRL. Downstream signalling may occur from the GITRL-associated receptor or from the GITRL itself. Such signalling and substances are described in more detail below.

In one embodiment of the invention activation of a GITRL pathway is achieved by administration of a GITR-ligand or an analogue thereof or an agonist of a GITRL-associated receptor.

The mouse and human GITRL genes are described in the NCBI database Entrez Gene at GeneID: 240873 and GeneID: 8995 respectively. The amino acid sequences of the human and mouse GITR-ligand (GITRL) are set out herein as SEQ ID NOS: 1 and 3. In accordance with the invention, a protein having SEQ ID NO: 1 or 3, or respective conservatively modified variants thereof, is administered to an individual for the prevention of treatment of a condition selected from type 2 diabetes, obesity and metabolic syndrome.

The term ‘conservatively modified variants’ is one well known in the art and indicates variants containing changes which are substantially without effect on antibody-antigen affinity. For example, it includes polypeptide or amino acid sequences which are derived from a particular starting polypeptide or amino acid sequence and which share a sequence identity that is about 80%, 85%, 86%, 87%, 88%, 89%, 90%, 91%, 92%, 93%, 94%, 95%, 96%, 97%, 98%, 99%, with the particular starting polypeptide or amino acid sequence. The term is also conveniently defined as found in U.S. Pat. No. 5,380,712 which is incorporated herein by reference for such purpose. It is well recognised in the art that the replacement of one amino acid in a peptide or protein with another amino acid having similar properties, for example the replacement of a glutamic acid residue with an aspartic acid residue, may not substantially alter the properties or structure of the peptide or protein in which the substitution or substitutions were made.

In another embodiment, a nucleic acid having SEQ ID NO: 2, 4 or 5, or a nucleic acid encoding a protein having SEQ ID NO: 1 or 3, or respective conservatively modified variants thereof, is administered to an individual for the prevention of treatment of a condition selected from type 2 diabetes, obesity and metabolic syndrome.

An analogue of a GITR-ligand has a similar structure and similar physical, chemical, biochemical, or pharmacological properties to GITR-ligand. Specifically in the context of the invention a GITR-ligand analogue will be sufficiently similar to GITR-ligand that it is able to activate a GITRL pathway.

An agonist of a GITRL-associated receptor is a substance that binds to a receptor of the GITR-ligand and triggers a response, thereby mimicking the action of a GITR-ligand. Preferably the agonist specifically binds to a GITRL-associated receptor, such as GITR. Mimicking the action of GITRL may result in GITRL-mediated signalling as described herein.

In particular, an analogue of GITRL or an agonist of a GITRL-associated receptor may bind to a GITRL-associated receptor and provide additive or synergistic stimulation (e.g. measures of T-cell proliferation) with cross-linking of the T-cell receptor. In industrial terms, this could be measured in a cell transfected a GITRL-associated receptor and with a reporter system associated with its signalling mechanism. Activation of the signalling mechanism by the agonist can be quantitated by the reporter system, for example an agonist antibody or expressed GITRL.

In one embodiment the GITRL-associated receptor is GITR. The agonist may comprise a small drug agonist or an antibody specific for a GITRL-associated receptor, such as an anti-GITR antibody.

The GITR-ligand or analogue thereof or agonist of a GITRL-associated receptor may comprise a GITR binding molecule or an antigen-binding fragment thereof. The term ‘GITR binding molecule’ refers to a molecule that specifically binds to GITR or a GITRL-associated receptor (i.e. another receptor of the GITR-ligand), preferably human GITR. GITR binding molecules for use in the methods of the invention include binding molecules that specifically bind to GITR and act as a GITR agonist (as demonstrated by, e.g., increased effector T cell response and/or increased humoral immunity), such as, for example, those binding molecules described in US20070098719, US20050014224, and WO05007190.

Preferred binding molecules according to the present invention are such that the affinity constant for the GITR antigen is 10⁵ mole⁻¹ or more, for example up to 10¹² mole⁻¹. Ligands of different affinities may be suitable for different uses so that, for example, an affinity of 10⁶, 10⁷, 10⁸, 10⁹, 10¹⁰ or 10¹¹ mole⁻¹ or more may be appropriate in some cases. However binding molecules with an affinity in the range of 10⁶ to 10⁸ mole⁻¹ will often be suitable. Conveniently the binding molecules also do not exhibit any substantial binding affinity for other antigens. Binding affinities of the binding molecules and binding specificity may be tested by assay procedures such as radiolabelled or enzyme labelled binding assays and use of biacore with solid phase ligand. (Bindon, C. I. et al (1988) Eur. J. Immunol. 18, 1507-1514; Dall'Acqua, W., et al Biochemistry 35, 9667; Luo et al J. Immunological Methods 275 (2203) 31-40 ; Murphy et al. Curr Protoc Protein Sci. 2006 Sep; Chapter 19: Unit 19.14).

Preferably the GITR binding molecule or an antigen-binding fragment thereof is an antibody or antibody fragment thereof.

### Antibody

The term ‘antibody’ as used herein includes all forms of antibodies such as recombinant antibodies, humanized antibodies, chimeric antibodies, single chain antibodies, monoclonal antibodies etc. The invention is also applicable to antibody fragments and derivatives that are capable of binding to the antigen.

Antibodies consist of two heavy and two light chains, each containing constant and variable regions. Limited proteolytic digestion with papain cleaves the antibody into three fragments. Two identical amino terminal fragments, each containing one entire light chain and about half a heavy chain, are the antigen binding fragments (Fab). This is the region of the antibody which recognises and binds an antigen. The sites of closest contact between antibody and antigen are the complementarity determining regions (CDR) of the antibody. The third fragment, similar in size but containing the carboxyl terminal half of both heavy chains with their interchain disulfide bond, is the crystallisable fragment (Fc). This is the region of the antibody which plays a role in modulating immune cell activity. The Fc contains carbohydrates, complement-binding, and FcR-binding sites. Limited pepsin digestion of the antibody yields a single F(ab′)2 fragment containing both Fab pieces and the hinge region, including the interchain disulfide bond between the two heavy chains. F(ab′)2 is divalent for antigen binding.

Chimeric antibodies are antibodies in which the whole of the variable regions of (for example) a mouse or rat antibody are expressed along with human constant regions. Humanised antibodies are effectively human antibodies in which only the complimentarity determining regions are derived from the rodent antibody variable regions and these are combined with framework regions from human variable regions.

The term ‘antibody’, as used herein, includes whole antibodies, e.g., of any isotype (IgG, IgA, IgM, IgE, etc.), and includes antigen binding fragments thereof. Exemplary antibodies include monoclonal antibodies, polyclonal antibodies, chimeric antibodies, humanized antibodies, human antibodies, and multivalent antibodies. Antibodies may be fragmented using conventional techniques. Thus, the term antibody includes segments of proteolytically-cleaved or recombinantly-prepared portions of an antibody molecule that are capable of actively binding to a certain antigen. Non-limiting examples of proteolytic and/or recombinant antigen binding fragments include Fab, F(ab′)2, Fab′, Fv, and single chain antibodies (sFv) containing a V[L] and/or V[H] domain joined by a peptide linker. The antibody may contain mutations in the Fc region which prevent binding of the antibody to Fc receptors. An example of a mutation in the Fc region which prevents binding of the antibody to Fc receptors is a mutation that prevents glycosylation of the antibody, for example an antibody in which the Fc region does not include a glycosylation site, for example an aglycosylated antibody.

The invention relates, in particular, to anti-GITR antibodies.

Anti-GITR antibodies suitable for use in accordance with the invention are described in the references cited herein and are known to the skilled person. Several anti-GITR antibodies are commercially available, e.g. mouse monoclonal anti-human GITR antibody (R&D Systems®, MAB689); rat monoclonal anti mouse-GITR (YGITR765) (Novus Biologicals®, NB100-64142) and mouse monoclonal anti human-GITR (1D8) (Novus Biologicals®, H00008784-MO2A). A humanised anti-GITR antibody is described in US 2009/0136494, which is incorporated herein by reference.

Alternatively the skilled person could make such anti-GITR antibodies by known methods. Various forms of anti-GITR antibodies can be made using standard recombinant DNA techniques (Winter and Milstein, Nature, 349, pp. 293-99 (1991)). For example, the production of rat proteolytic fragments of IgG antibodies is described by Rousseaux, J (Methods in Enzymology 1986; 121; 663). Antibodies: A Laboratory Manual (Ed Harlow, Edward Harlow, David Lane, CSHL Press, 1988) describes obtaining fragments of human antibodies. Gilliland et al (Tissue Antigens 1996; 47(1):1-20) describes a general method for isolating the variable regions of antibodies and the production of a chimeric antibody. The preparation of monoclonal antibodies is a well-known process (Kohler et al., Nature, 256:495 (1975)).

Chimeric and humanised, e. g. CDR-grafted, antibodies may be used in accordance with the present invention. These antibodies are less immunogenic than the corresponding rodent antibodies. Thus, the antibody may have CDRs which are of different origin to the variable framework region. Similarly, the antibody may have CDRs of different origin to the constant region.

The antibody may have variable domain framework regions which are of human, rat, hamster or mouse origin or are derived from those of human, rat, hamster or mouse origin.

The antibody may have constant domains which are of human, rat, hamster or mouse origin or are derived from those of human, rat, hamster or mouse origin. For chimeric and humanised antibodies the constant domains will be of human origin or derived from human origin. The antibody may have a constant region which is of an IgG isotype. The antibody may have CDRs which are of human, rat, hamster or mouse origin or are derived from those of human, rat, hamster or mouse origin. The antibody may have only one of its arms with an affinity for the GITR antigen. The antibody may be monovalent. The antibody may have one half of the antibody consisting of a complete heavy chain and light chain and the other half consisting of a similar but truncated heavy chain lacking the binding site for the light chain.

In preferred embodiments the antibody is a monoclonal antibody, preferably an aglycosylated IgG antibody. Preferably the aglycosylated antibody has a binding affinity for the human GITR antigen. The antibody may be a chimeric or humanized antibody or a fragment thereof. For example, the antibody may consist of a chimeric antibody having the one or more (e.g. 2, 3, 4, 5 or 6) of the following CDRs, or respective conservatively modified variants thereof (as defined above):

(a)
(Heavy Chain CDR1 - SEQ ID NO: 6)
GFSLSTSGMGVG
(b)
(Heavy Chain CDR2N - SEQ ID NO: 7)
HIWWDDDKYYNPSLKS
(b)ii
(Heavy Chain CDR2Q - SEQ ID NO: 8)
HIWWDDDKYYQPSLKS
(c)
(Heavy Chain CDR3 - SEQ ID NO: 9)
TRRYFPFAY
(d)
(Light Chain CDR1 - SEQ ID NO: 10)
KASQNVGTNVA
(e)
(Light Chain CDR2 - SEQ ID NO: 11)
SASYRYS
(f)
(Light Chain CDR3 - SEQ ID NO: 12)
QQYNTDPLT.

In a chimeric antibody the CDRs (a), (b) or (b)ii, and (c) are arranged in the heavy chain in the sequence in the order: rodent framework region 1/(a)/rodent framework region 2/(b) or (b)ii/rodent framework region 3/(c)/rodent framework region 4 in a leader to constant domain (N-terminal to C-terminal) direction and the CDRs (d), (e) and (f) are arranged in the light chain in the sequence: rodent framework region 1/(d)/rodent framework region 2/(e)/rodent framework region 3/(f)/rodent framework region 4 in a leader to constant domain direction. It is preferred, therefore, that where all three are present the heavy chain CDRs are arranged in the sequence (a), (b) or (b)ii, (c) in a leader to constant domain direction and the light chain CDRs are arranged in the sequence (d), (e), (f) in a leader to constant domain direction. The rodent framework region is preferably rat but may also be mouse.

In a humanized antibody the above framework sequences would be of human origin.

It should be appreciated however, that antibodies according to the invention may contain quite different CDRs from those described hereinbefore and that, even when this is not the case, it may be possible to have heavy chains and particularly light chains containing only one or two of the CDRs (a), (b), (b)ii and (c) and (d), (e) and (f), respectively. However, although the presence of all six CDRs defined above is therefore not necessarily required in an antibody according to the present invention, all six CDRs will most usually be present in the most preferred antibodies.

It is well recognised in the art that the replacement of one amino acid in a CDR with another amino acid having similar properties, for example the replacement of a glutamic acid residue with an aspartic acid residue, may not substantially alter the properties or structure of the peptide or protein in which the substitution or substitutions were made. Thus, an aglycosylated antibody may include those antibodies containing the preferred CDRs but with a specified amino acid sequence in which such a substitution or substitutions have occurred without substantially altering the binding affinity and specificity of the CDRs. Alternatively, deletions may be made in the amino acid residue sequence of the CDRs or the sequences may be extended at one or both of the N- and C-termini whilst still retaining activity.

Preferred antibodies according to the present invention are such that the affinity constant for the antigen is 10⁵ mole⁻¹ or more, for example up to 10¹² mole⁻¹. Ligands of different affinities may be suitable for different uses so that, for example, an affinity of 10⁶, 10⁷, 10⁸, 10⁹, 10¹⁰ or 10¹¹ mole⁻¹ or more may be appropriate in some cases. However antibodies with an affinity in the range of 10⁶ to 10⁸ mole⁻¹ will often be suitable. Conveniently the antibodies also do not exhibit any substantial binding affinity for other antigens. Binding affinities of the antibody and antibody specificity may be tested by assay procedures such as radiolabelled or enzyme labelled binding assays and use of biacore with solid phase ligand. (Bindon, C. I. et al. 1988 Eur. J. Immunol. 18, 1507-1514; Dall'Acqua, W., et al. Biochemistry 35, 9667; Luo et al. J. Immunological Methods 275 (2203) 31-40; Murphy et al. Curr Protoc Protein Sci. 2006 Sep; Chapter 19: Unit 19.14).

Of the CDRs it is the heavy chain CDRs (a), (b) and (c) that are of most importance. It will be realised by those skilled in the art that the antibodies of the invention also comprise constant domains. The constant domains are preferably of human origin.

### Administration

In the treatment using a GITR-ligand or an analogue thereof or an agonist of a GITRL-associated receptor, such substance may be used alone or may be mixed with a pharmaceutically acceptable carrier or diluent by a method commonly used depending on an administration route, so as to manufacture a pharmaceutical composition having a suitable dosage form. Any appropriate carrier or diluent may be used, for example isotonic saline solution, buffers, etc. Such pharmaceutical carriers are well known in the art and the selection of a suitable carrier is deemed to be within the scope of those skilled in the art from the teachings contained herein. The ratio of an active ingredient to a carrier can be changed between 1% and 90% by weight. The pharmaceutical composition of the present invention may be administered to humans or organisms other than the humans [for example, non-human mammals (e.g. a bovine, a monkey, a chicken, a cat, a mouse, a rat, a hamster, a swine, a canine, etc.), birds, reptiles, amphibians, fish, insects, etc.]. Any dosage form known to the skilled person may be used. Accordingly, the pharmaceutical composition of the present invention may be administered via either an oral administration route or a parenteral administration route (e.g. intravenous injection, intramuscular injection, subcutaneous administration, rectal administration, and dermal administration). Antibodies are generally given by injection or by infusion.

The GITR-ligand or an analogue thereof or an agonist of a GITRL-associated receptor is administered in vivo in an amount effective to activate a GITRL pathway and produce the desired effect in terms of preventing or treating one or more of the conditions listed herein. The desired effect may be the alleviation or inhibition of one or more symptoms of the condition, prevention of the development of the condition, or regression of the condition, for example. Key identifiers of the desired effect include a reduction in weight gain, fatty liver, or levels of ALT, AST, lactate dehydrogenase, cholesterol, glucose, insulin, free fatty acids (FFAs), ketone bodies (e.g. BHBA) or interleukin-6 in the blood. Other identifiers of the desired effect may be a return to normal levels of one or more of triglycerides, essential fatty acids (e.g. eicosapentaenoate, docosapentaenoate, dihomo-linolenate and docosahexaenoate), arginine, proline, ornithine, trans-4-hydroxyproline, corticosterone, arachnidonate or citrulline, as discussed in more detail below. The term ‘an effective amount’ for purposes of this application shall mean that amount of substance capable of producing the desired effect. The amount of substance which is given depends upon a variety of factors including the age, weight and condition of the patient, the administration route, the properties of the pharmaceutical composition, the condition of the patient, the judgment of a doctor, the condition and the extent of treatment, prevention or control desired. In a mouse model system, doses of between 1 mg and 100 mg/kg, preferably between 20 and 60 mg/kg, of an anti-GITR antibody may be given. In humans, between 10 ug and 10 mg/kg, preferably between 100 ug and 1 mg/kg, may be appropriate. In this sense, ‘dose’ refers to the total amount of antibody administered over a 24 hour period.

The substance may be administered to the individual as a short-term therapy intended to induce tolerance and with the aim of achieving long-term benefit. For this type of therapy it is preferred to use an antibody. For example, the substance (e.g. antibody) may be administered to the patient in an effective amount over a period of up to 4 weeks without subsequent re-administration after this period for at least 6 months. Preferably the substance is administered over a period of up to 2 weeks and most preferably for 1, 2, 3, 4 or 5 days. The substance may be administered daily, on alternate days, 3-4 times a week or weekly for a period of 1, 2, 3 or 4 weeks. The substance may be administered once or more than once on each day of administration.

Preferably there is no subsequent re-administration after this period for at least 12 months, at least 2 years or most preferably at least 3, 4 or 5 years. However, if needed, booster or reinforcing doses of the substance may be given but such booster doses should not be required more often than once every 6 months, preferably not more than once every year or every 2 years.

Alternatively, the substance may be administered to the individual as a long-term therapy intended to prevent or control the condition.

The GITR-ligand or an analogue thereof or an agonist of a GITRL-associated receptor may be employed alone or in combination with other techniques, drugs or compounds for preventing, controlling or treating obesity, metabolic syndrome or type 2 diabetes. For example, the GITR-ligand or an analogue thereof or an agonist of a GITRL-associated receptor may be administered in combination (e.g. contemporaneously or sequentially) with one or more of the following: appetite suppressants (such as phentermine, sibutramine and orlistat); sulphonylureas (such as chlorpropamide, tolbutamide, glyburide, glipizide and glimepiride); meglitinides (such as repaglinide and nateglinide); biguanides (such as biguanide metformin); thiazolidinediones (such as pioglitazone and rosiglitazone); insulin; alpha glucosidase inhibitors (such as acarbose); anti-hyperglycemic medications (such as pramlintide); DPP IV inhibitors (such as sitagliptin and saxagliptin); and ACE inhibitors (such as benazepril, captopril, enalapril, perindopril and ramipril).

In the gene therapy of the present invention, it may be possible to select either an in vivo method of directly administering a recombinant vector encoding the gene of interest to a patient, or an ex vivo method of collecting a target cell from a patient body, introducing a GITRL gene, or a recombinant vector encoding the gene of interest, or DNA constructs carrying agonist encoding genes e.g. heavy and light chains of an engineered agonist antibody, into the target cell outside of the body, and returning the target cell, into which the aforementioned gene or vector has been introduced, to the patient body. In the case of the in vivo method, the recombinant vector encoding the gene of interest is directly administered to a patient by using a gene transfer vector known in the present technical field, such as a retrovirus vector. As with the pharmaceutical composition of the present invention, such a GITRL gene used in the gene therapy of the present invention, or a gene transfer vector to which the GITRL- or other GITR agonist-encoding gene is operably linked, can be mixed with a pharmaceutically acceptable carrier, so as to produce a formulation. Such a formulation can be parenterally administered, for example. Fluctuation of a dosage level can be adjusted by standard empirical optimizing procedures, which are well understood in the present technical field. An alternative for in vivo administration is to use physical approaches, such as particle bombardment or jet injection, to directly deliver DNA encoding heavy and light chains of an engineered agonist antibody (Walther et at Mole Biotechnology 28:121-128, 2004; Yang et at PNAS 87:9568-72, 1990). In the case of the ex vivo method, such a GITRL gene or other GITR agonist gene can be introduced into a target cell according to a method known in the present technical field, such as the calcium phosphate method, the electroporation method, or the viral transduction method. Such a target cell can be collected from the affected region of a cerebral nerve system or the like. In the case of selecting the ex vivo method, a GITRL (or GITR agonist) gene or a gene transfer vector to which the GITRL (or GITR agonist) gene is operably linked is introduced into a cell, and the aforementioned peptide is then allowed to express in the cell. Thereafter, the cell is transplanted to a patient, so that a nerve-related disease caused by inactivation of the GITRL gene can be treated.

A gene transfer vector available for gene therapy is well known in the present technical field, and it can be selected, as appropriate, depending on a gene introduction method or a host. Examples of such a vector include an adenovirus vector and a retrovirus vector. When a GITRL gene is ligated to a gene transfer vector, a control sequence such as a promoter or a terminator, a signal sequence, a polypeptide-stabilizing sequence, etc. may be appropriately ligated, such that the gene can be expressed in a host. Selection or construction of such vectors is well known to the skilled person.

### Screening Method

In the examples below a Gitrl−/− mouse is described which has a phenotype of increased weight, fatty liver and increased levels of ALT, AST, lactate dehydrogenase and blood glucose, insulin, cholesterol, free fatty acids (FFAs), ketone bodies (e.g. BHBA) and IL6. Since this animal has a homozygous disruption of the GITRL gene, it is considered that an obese phenotype is caused by inactivation of the GITRL gene. Accordingly, the non-human gene-disrupted (Gitrl−/−) animal can be used as a model animal of obesity caused by inactivation of the GITRL gene. In particular, the above non-human gene-disrupted animal can be used in the searching and development of a therapeutic agent for obesity and the related conditions metabolic syndrome and type 2 diabetes.

Accordingly, in another aspect the invention provides a screening method for identifying substances useful in the prevention, control or treatment of conditions such as type 2 diabetes, obesity and metabolic syndrome. Specifically the invention provides a method for screening for a substance, or a salt or a solvate thereof, to be used in the prevention or treatment of a condition selected from type 2 diabetes, obesity and metabolic syndrome, which comprises the following steps:

(i) measuring the severity of a symptom or sign of the condition in a non-human animal in which the GITRL gene is disrupted;

(ii) administering a test substance to said non-human animal;

(iii) measuring the severity of a symptom or sign of the condition of said non-human animal after administration of the test substance; and

(iv) comparing the severity of the symptom or sign of the condition before administration of the test substance with the severity of the symptom or sign of the condition after administration of the test substance, wherein a decrease or lessening of the symptom or sign indicates that the substance administered may be useful in the prevention or treatment of the condition.

The non-human animal in which the GITRL gene is disrupted may be a rodent, for example a rat or a mouse.

In the screening method of the present invention, the severity of a symptom of obesity, metabolic syndrome or type 2 diabetes of a gene-disrupted animal before administration of a test substance is compared with the severity of the symptom of the obesity, metabolic syndrome or type 2 diabetes of the gene-disrupted animal after administration of the test substance. When the symptom(s) are reduced in number or severity as compared to the former levels, it can be determined that the test substance is useful for the treatment of the disease. The symptom or sign to be measured may be one or more of weight gain, fatty liver, or levels of ALT, AST, lactate dehydrogenase, cholesterol, glucose, insulin, free fatty acids (FFAs), ketone bodies (e.g. BHBA), or interleukin-6 in the blood, as discussed above. Additional signs to be measured may include the levels of one or more biomarkers selected from triglycerides, essential fatty acids (e.g. eicosapentaenoate, docosapentaenoate, dihomo-linolenate and docosahexaenoate), arginine, proline, ornithine, trans-4-hydroxyproline, corticosterone or arachnidonate in the blood, whereby the effectiveness of the test substance would be determined by a return to normal levels of the biomarker, as discussed in more detail below. Combining information from two or more of these symptoms or signs may be confirmative.

The type of a substance screened by the screening method of the present invention is not particularly limited. Examples of such a substance include a therapeutic agent for treating obesity, metabolic syndrome or type 2 diabetes or a candidate compound therefor.

The test substance should be administered to the non-human animal using a dosage regime which may be established by standard experimentation, as will be apparent to the skilled person.

The technique of disrupting a gene is known to persons skilled in the art and they will be able to disrupt a gene of interest according to known methods. For example, the GITRL gene can be disrupted by insertion of a foreign sequence into the GITRL gene, by substitution of the entire or a part of the GITRL gene with a foreign sequence, or by deletion of the entire or a part of the GITRL gene. The number of bases of such a foreign sequence and the position of the GITRL gene to be substituted, deleted or inserted are not particularly limited, as long as expression of the GITRL protein or the activity thereof is substantially lost. From the viewpoint of selection of a recombinant gene, such a foreign sequence is preferably a selective marker gene. Such a selective marker gene can be appropriately selected from known selective marker genes, such as drug resistance genes such as a neomycin resistance gene or a puromycin resistance gene. Moreover, the GITRL gene can also be disrupted by introducing a mutation such as a deletion, insertion, or substitution in the above GITRL gene. For example, a mutation that has a fatal influence (loss of expression or activity) on the functions of a protein, such as a frameshift mutation or a nonsense mutation, can be introduced into the aforementioned gene. Alternatively, the GITRL gene can be disrupted by targeted disruption. All of these techniques are well-known to the skilled person.

Steps for the production of a Gitrl−/− may include the following.

### (i) Transformation of ES Cells

DNA encoding the full or partial GITRL gene is obtained and inserted into a vector construct also containing a drug resistance gene. The vector may preferably include a negative selective marker such as a thymidine kinase gene or a diphtheria toxin gene. Any type of vector can be used, as long as it can autonomously replicate in cells to be transformed (e.g. Escherichia coli). For example, commercially available pBluescript (Stratagene), pZErO1.1 (Invitrogen), pGEM-1 (Promega), etc. can be used.

### (ii) Selection of Transformed ES Cells

The produced targeting vector is cleaved with restriction enzymes to form linear DNA, which is purified and transfected into ES cells (e.g. by electroporation or lipofection). The transfected ES cells are cultured in a suitable selective medium and incorporation of the GITRL gene is determined (e.g. by Southern blot and/or PCR). Southern blot analysis is used to determine whether homologous recombination has occurred and to confirm that the targeting vector has not been randomly inserted. These methods are used in combination, so as to obtain homologous recombinant ES cells.
(iii) Introduction of ES Cells Into Embryo or Blastocyst
A GITRL gene knockout mouse can be produced by such steps as collection of an 8-cell-stage embryo or blastocyst after fertilization, microinjection of homologous recombinant ES cells, transplantation of a manipulated egg into a pseudopregnant mouse, the delivery of the pseudopregnant mouse and breeding of born babies, selection of the gene-introduced mouse by the PCR method and the Southern blotting method, and the establishment of a mouse line having the introduced gene (Yagi, T. et. al., Analytical Biochem. 214, 70, 1993).

### (iv) Transplantation of Manipulated Egg Into Pseudopregnant Mouse and Establishment of Heterozygous Mouse

A vasoligated male mouse may be crossed with a normal female mouse to produce a pseudopregnant mouse. A manipulated egg is then transplanted into the pseudopregnant mouse. Transplantation of such a manipulated egg is carried out, based on the descriptions of Hogan, B. L. M., A Laboratory Manual, Cold Spring Harbor Laboratory Press, Cold Spring Harbor, N.Y., 1986, or Yagi T. et. al., Analytical Biochem. 214, 70, 1993.

Whether or not the introduced gene has been incorporated into germ cells can be easily confirmed by crossing a mouse to be examined with a mouse with white hair (e.g. ICR) and observing the hair color of the obtained baby mice. Since it is anticipated that a mouse having a high chimeric rate contains the introduced gene in the germ cells thereof, it is preferable to select a mouse having a chimeric rate that is as high as possible.

The obtained chimeric mouse is crossed with a wild-type mouse (normal mouse), so as to obtain a heterozygous mouse (hereinafter referred to as a “hetero mouse” at times). DNA is extracted from an ear clip of the obtained baby mouse, and the presence or absence of the introduced gene can be then confirmed by the PCR method. In addition, instead of the PCR method, the Southern blot analysis can be applied to more reliably identify a genotype.

### (v) Establishment of Homozygous Mouse Line

When the two heterozygous mice are crossed to obtain baby mice, GITRL gene knockout mice wherein the introduced gene exists in a homozygous manner (hereinafter referred to as homozygous mice) can be obtained. Such a GITRL knockout mouse can be obtained by any one of the crossing of the two heterozygous mice, the crossing of the heterozygous mouse with the GITRL gene knockout mouse, and the crossing of the two GITRL gene knockout mice. The presence or absence of expression of the mRNA of such GITRL gene knockout mouse can be confirmed by the Northern blot analysis, the RT-PCR method, the RNAse protection assay, the in situ analysis, etc. Moreover, presence or absence of expression of GITRL protein can be confirmed by immunohistological staining, the use of an antibody that recognizes the aforementioned protein, etc.

### Predictive and Diagnostic Biomarkers

According to yet another aspect the invention provides a method for diagnosing or predicting the risk of developing a condition selected from type 2 diabetes, obesity and metabolic syndrome in a subject, which comprises measuring the concentration of one or more biomarkers in the blood of or in a plasma sample from the subject, wherein the one or more biomarkers are selected from ALT, AST, lactate dehydrogenase, cholesterol, glucose, insulin, free fatty acids (FFAs), ketone bodies (e.g. BHBA), interleukin-6, triglycerides, essential fatty acids (e.g. eicosapentaenoate, docosapentaenoate, dihomo-linolenate and docosahexaenoate), arginine, proline, ornithine, trans-4-hydroxyproline, corticosterone, arachnidonate or citrulline. The concentration of the measured biomarker may be compared to the concentration of the biomarker in a healthy subject, wherein a change in the level of the biomarker is indicative of one or more of the conditions. Alternatively or additionally the concentration of the measured biomarker may be measured repeatedly in the subject over a period of time, wherein a change in the level of the biomarker over time is indicative of one or more of the conditions. The biomarker concentration may be measured over a series of timepoints, for example weekly, fornightly or monthly, and over a period of time, for example from 1 month to 1 year, preferably 1, 2, 3, 4, 5 or 6 months.

The biomarker may be selected from ALT, AST, lactate dehydrogenase, cholesterol, glucose, insulin, free fatty acids (FFAs), ketone bodies (e.g. BHBA), interleukin-6, triglycerides, essential fatty acids (e.g. eicosapentaenoate, docosapentaenoate, dihomo-linolenate and docosahexaenoate), arginine, proline, ornithine, trans-4-hydroxyproline, corticosterone, arachnidonate or citrulline. For each biomarker, diagnosis of or prediction of the risk of developing a condition selected from type 2 diabetes, obesity and metabolic syndrome in a subject may be determined as set out below.

Elevated levels of liver enzymes, such as ALT and AST, are indicative of a risk of developing the condition or of the condition itself. In contrast constant levels are indicative of a healthy subject.

Elevated levels of lactate dehydrogenase are indicative of a risk of developing the condition or of the condition itself In contrast constant levels are indicative of a healthy subject.

Increasing levels of IL6 over time are indicative of a risk of developing the condition or of the condition itself In contrast constant levels of IL6 are indicative of a healthy subject.

Elevated insulin is indicative of a risk of developing the condition or of the condition itself In contrast constant levels are indicative of a healthy subject.

Elevated triglycerides are indicative of a risk of developing the condition or of the condition itself In contrast constant levels are indicative of a healthy subject.

High levels of cholesterol are indicative of a risk of developing the condition or of the condition itself In contrast a lower constant level is indicative of a healthy subject.

Raised levels of glucose early in a time course are indicative of a risk of developing the condition or of the condition itself In contrast increasing or constant levels are indicative of a healthy subject.

Decreasing levels of ketone bodies or in particular BHBA over a time course are indicative of a risk of developing the condition or of the condition itself. In contrast a constant lower level is indicative of a healthy subject.

Decreasing levels of FFAs, essential fatty acids or in particular eicosapentaenoate, docosapentaenoate, dihomo-linolenate and docosahexaenoate over a time course are indicative of a risk of developing the condition or of the condition itself In contrast increasing or constant levels are indicative of a healthy subject.

Increasing levels of arginine over a time course are indicative of a risk of developing the condition or of the condition itself. In contrast slightly decreasing or constant levels are indicative of a healthy subject.

Decreasing levels of citrulline over a time course are indicative of a risk of developing the condition or of the condition itself. In contrast slightly decreasing or constant levels are indicative of a healthy subject.

Increasing levels of proline over a time course are indicative of a risk of developing the condition or of the condition itself. In contrast decreasing levels are indicative of a healthy subject.

Increasing levels of ornithine over a time course are indicative of a risk of developing the condition or of the condition itself. In contrast slightly decreasing or constant levels are indicative of a healthy subject.

Increasing levels of trans-4-hydroxyproline over a time course are indicative of a risk of developing the condition or of the condition itself. In contrast slightly decreasing or constant levels are indicative of a healthy subject.

Decreasing levels of corticosterone over a time course are indicative of a risk of developing the condition or of the condition itself. In contrast increasing or constant levels are indicative of a healthy subject.

Decreasing levels of arachidonate over a time course are indicative of a risk of developing the condition or of the condition itself. In contrast an increasing level is indicative of a healthy subject.

In some embodiments a single biomarker may be sufficient to enable the diagnosis or prediction of the risk of developing a condition. For example an increased concentration of one or more of cholesterol, insulin, glucose and IL6 as compared to a healthy subject is indicative of disease-associated metabolic and inflammatory events. These are considered as good primary endpoints for test treatments.

An additional or alternative approach may be a systems biology approach in which the levels of two or more, preferably, 2, 3, 4, 5, 6, 7, 8, 9, 10 or more biomarkers are measured. Preferably such biomarkers are measured in a subject over time as discussed above. This could be important because reverse trends in may be observed in healthy subjects and patients as discussed above. For example decreasing levels of essential fatty acids (e.g. eicosapentaenoate, docosapentaenoate, dihomo-linolenate and docosahexaenoate) or ketone bodies (e.g. BHBA) in a subject over time is indicative of disease-associated metabolic and inflammatory events. Similarly decreases over time in levels citrulline, corticosterone or arachnidonate and/or increases over time in levels of arginine, proline, ornithine or trans-4-hydroxyproline, may be indicative of type 2 diabetes, obesity and metabolic syndrome. This “systems biology” approach in which two or more biomarkers are measured has the advantage that the prediction or diagnosis can be made with increased certainty based on the combination of biomarkers.

When diagnosing or predicting the risk of developing a condition it is important to exclude other possible diagnoses. For example, increased levels of IL6 over time are indicative of a risk of developing metabolic syndrome, but may also be observed in forms of inflammation. The skilled person, when making such a prediction or diagnosis, will be aware of such circumstances and using his knowledge will exclude such other diagnoses. For example, this may be done by making use of others symptoms, signs or biomarkers, since in combination with the biomarker in question they can enable the diagnosis of early disease risk. In this respect it is preferable that different sets, or fingerprints, of markers may be most useful in arriving at a specific diagnosis or prediction. For example, if a patient has high IL6 with no indications of an acute or chronic inflammation elsewhere, coupled with changes in insulin levels and cholesterol, then this will constitute a more defined indicator of risk than the knowledge of high IL6 alone.

Such measurement of these biomarkers may allow early detection, diagnosis or prognosis of these conditions, before the clinical disease becomes obvious or symptoms become detectable. Advantageously, this may allow early changes in lifestyle or early preventative or therapeutic treatments, as well as an indication of treatment benefit.

The level of each biomarker in the plasma may be measured by any method known to the skilled person. Many such methods are well-known. For some biomarkers it may be possible to monitor or measure the level of the biomarker in vivo, for example by means of a sensor. The biomarker may also be monitored or measured by analysing blood or plasma samples taken from a subject under conditions suitable for the particular assay, as would be apparent to the skilled person. The level of the biomarker may be measured using chemical, biological or enzymatic methods or using antibodies, e.g. an immunoassay. The method may involve a test strip or dipstick, a meter, or the like, and may involve purification and detection or direct detection of the biomarker. Liquid chromatography, gas chromatography or mass spectrometry may be used (e.g. LC/MS, GC/MS, MS/MS, LC/MS/MS).

The invention also provides a method for monitoring the effectiveness of a treatment of a condition selected from type 2 diabetes, obesity and metabolic syndrome comprising measuring the concentration of one or more biomarkers in the blood of or in a plasma sample from the subject, wherein the one or more biomarkers are selected from ALT, AST, lactate dehydrogenase, cholesterol, glucose, insulin, free fatty acids (FFAs), ketone bodies (e.g. BHBA), interleukin-6, triglycerides, essential fatty acids (e.g. eicosapentaenoate, docosapentaenoate, dihomo-linolenate and docosahexaenoate), arginine, proline, ornithine, trans-4-hydroxyproline, corticosterone, arachnidonate or citrulline. These biomarkers may be monitored over time as discussed above, for example, prior to and during treatment, preferably at regular intervals such as weekly or monthly, whereby return or partial return to an accepted normal level is indicative of a beneficial or effective treatment.

The invention also provides a method for selecting an appropriate therapy for treating a condition selected from type 2 diabetes, obesity and metabolic syndrome in a patient comprising measuring the concentration of one or more biomarkers in the blood of or in a plasma sample from the subject, wherein the one or more biomarkers are selected from ALT, AST, lactate dehydrogenase, cholesterol, glucose, insulin, free fatty acids (FFAs), ketone bodies (e.g. BHBA), interleukin-6, triglycerides, essential fatty acids (e.g. eicosapentaenoate, docosapentaenoate, dihomo-linolenate and docosahexaenoate), arginine, proline, ornithine, trans-4-hydroxyproline, corticosterone, arachnidonate or citrulline. By monitoring the levels of metabolic markers and the levels of inflammatory markers it is possible to determine therapy with appropriate anti-inflammatory, metabolic control and lifestyle components. The biomarkers associated with metabolic changes include ALT, AST, lactate dehydrogenase, cholesterol, glucose, insulin, FFAs, ketone bodies (e.g. BHBA) free fatty acids - essential fatty acids (e.g. eicosapentaenoate, docosapentaenoate, dihomo-linolenate and docosahexaenoate. The biomarkers associated with inflammatory changes include IL6, corticosterone, arachnidonate, arginine and citrulline, proline, ornithine, trans-4-hydroxyproline. The expected levels and patterns of these biomarkers in healthy subjects and patients is discussed above.

### EXAMPLES

The following examples are illustrative of the products and methods falling within the scope of the present invention. They are not to be considered in any way limitative of the invention.

### Example 1

### Generation of Knockout Mouse Gitrl−/−

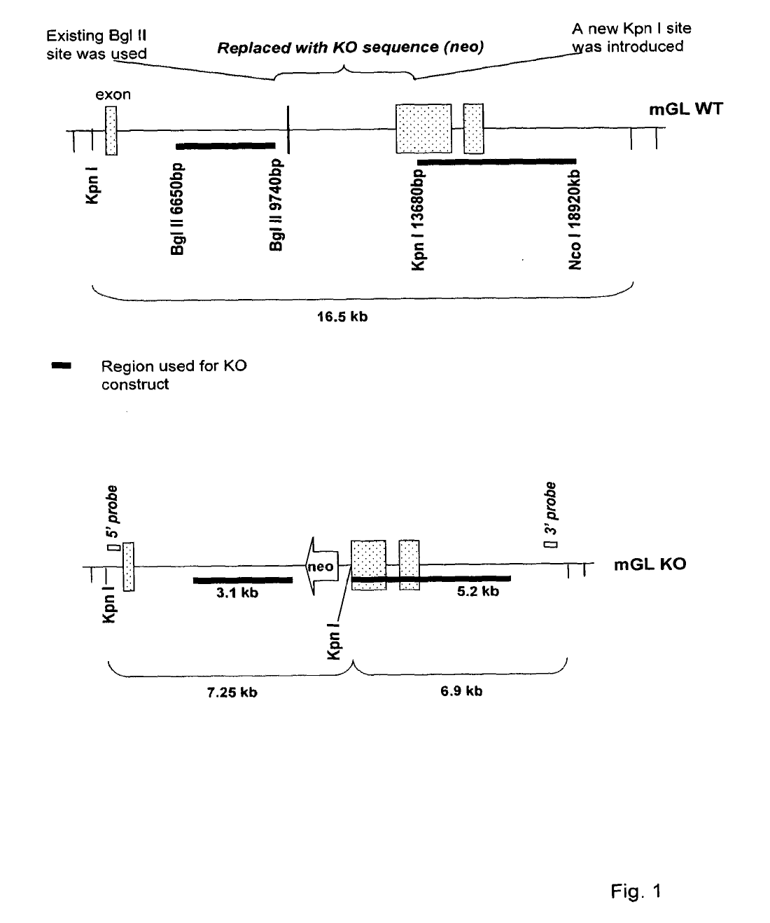

A knockout mouse was generated as shown in FIG. 1 which shows schematic representations of the GITRL gene locus in wild type and GITRL−/− knockout mice. Shaded areas represent the exons of the mouse GITRL gene. Horizontal bars represent the extent of the arms of the targerting construct used to replace exon 2 and the coding region of exon 3 of the wildtype gene with a neomycin cassette.

Knocking out GITRL in the 129/sv strain of mice was embryonic lethal in the homozygote. As the 129 GITRL mice were lethal as homozygotes, heterozygote GITRL−/− mice were crossed with C57/BL6J wild type mice. Heterozygote off-spring were identified by PCR of genomic DNA from ear clipped skin and these mice were crossed for a further generation with C57/BL6J wild type mice. Heterozygote offspring from the same generation were intercrossed and the numbers of homozygote Gitrl−/− mice assessed by PCR. Homozygous Gitrl−/− lethality was negated by backcrossing onto C57B1/6J strain, with Mendelian pup ratios observed by generation 9 (N9). A Gitrl−/− line was established using N9 founders.

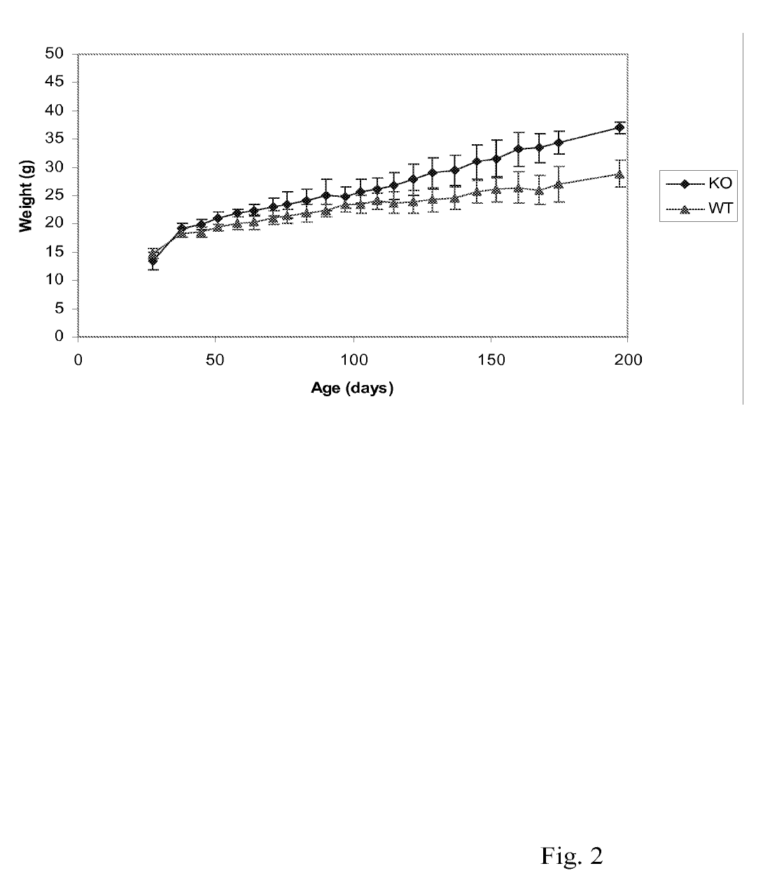

The Gitrl−/− animals were observed to be heavier than WT C57B1/6 mice of the same age. This increased weight was first noted in intercross pups at the second generation backcross and monitored for a period of 6 months at the sixth generation. FIG. 2 shows growth curves of female WT C57B1/6J (n=4) and homozygous 6 generation backcrossed Gitrl−/− (n=4) mice. It can be seen that the Gitrl−/− animals (KO, diamond symbols) were heavier than WT C57B1/6J mice (WT, triangle symbols) of the same age. The Gitrl−/− mice appeared to eat more, but also to gain more weight per gram of food consumed.

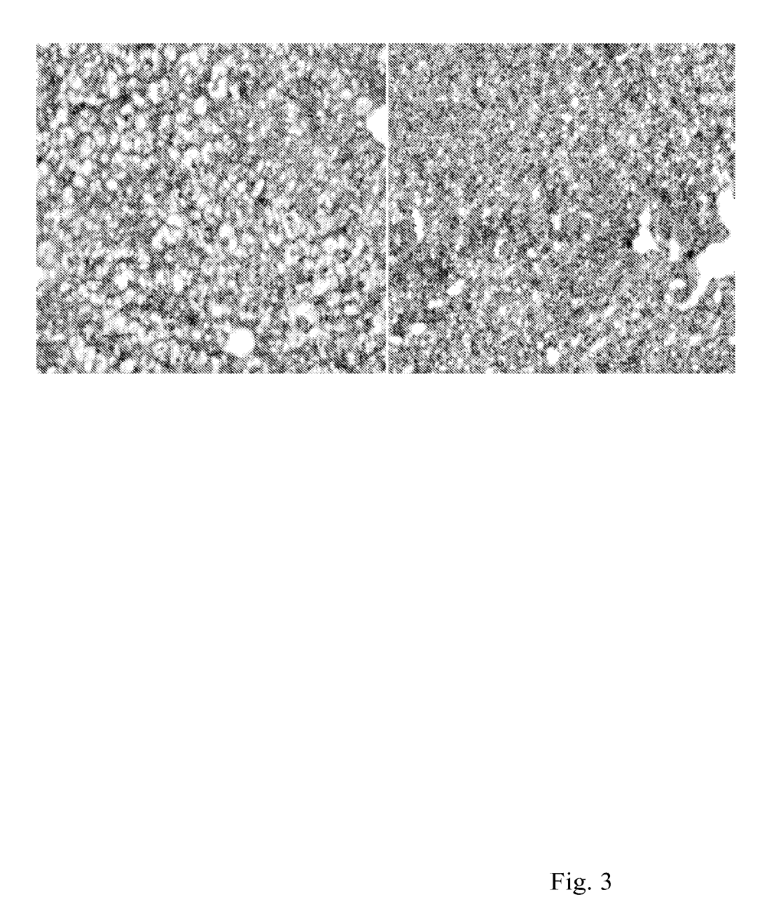

FIG. 3 shows oil red O staining of liver sections from 6 month old C57B1/6J WT (left) and 6 generation backcrossed Gitrl−/− (right) mice. It can be seen that at 6 months the mice had enlarged hearts and livers and showed hepatic fat accumulation.

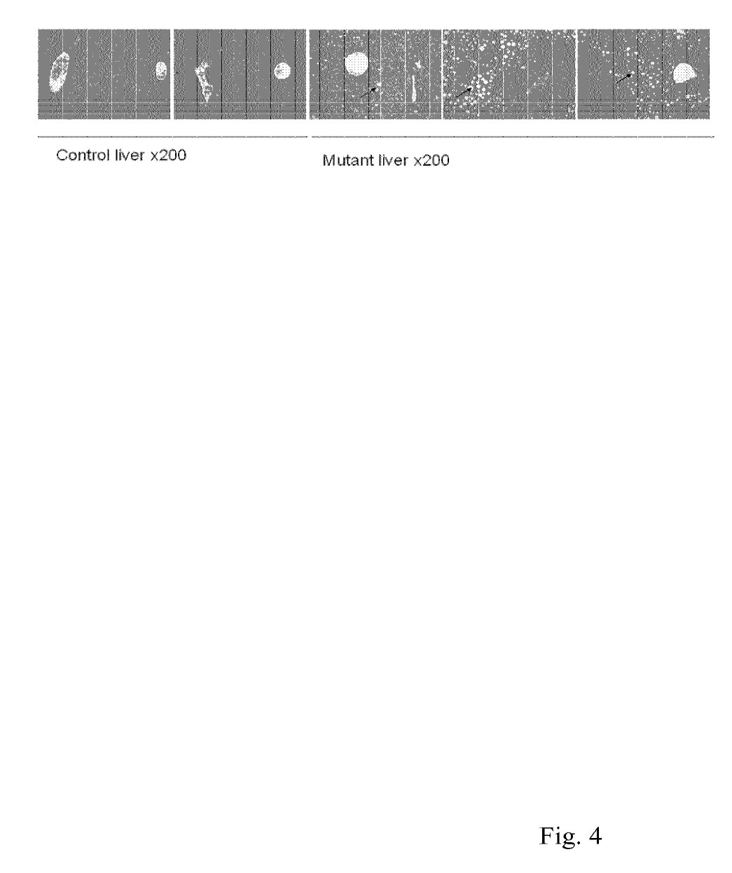

FIG. 4 shows hematoxylin and eosin staining of livers of 2 control (left panels) and 3 GITRL−/− mice (9th generation backcross) (right panels). Spaces (i.e. white/lighter areas) in pink staining liver sections show areas where lipid has been extracted by the solvents used in histological preparation illustrating the accumulation of fat in livers of mice lacking GITRL.

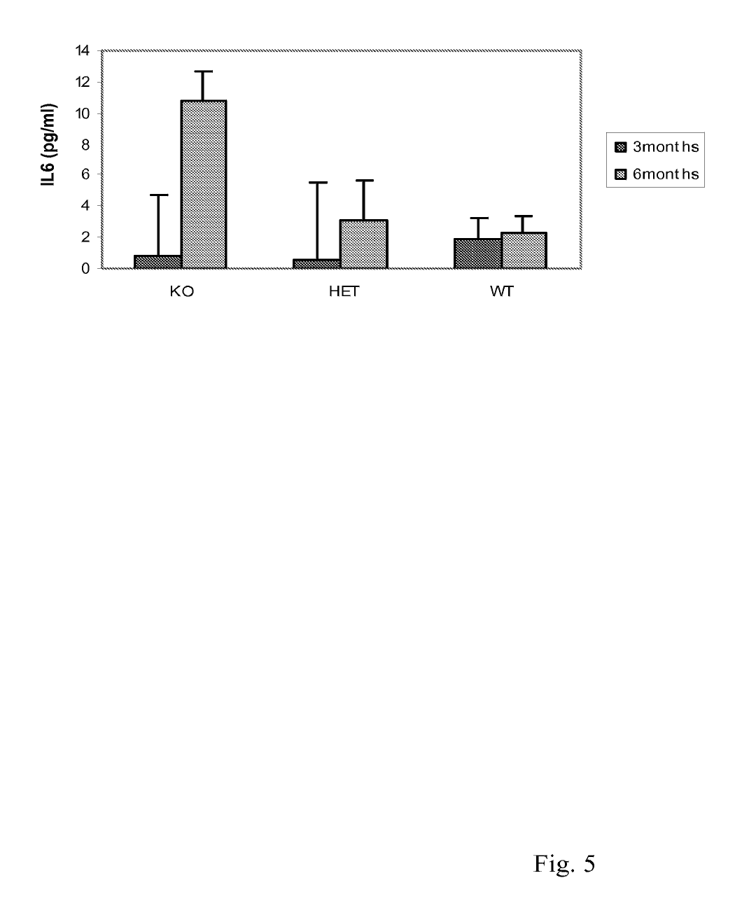

FIG. 5 shows serum IL-6 at 3 and 6 months in female C57B1/6J WT and 6 generation backcrossed Gitrl+/− and Gitrl−/− mice. Analysis of serum in the Gitrl+/− and Gitrl−/− mice revealed elevated IL6 at 6 months compared to the wild type mice. Over this time period (from 3 to 6 months), the levels of IL6 increased in the knockout mice whilst they remained constant in the wild type mice.

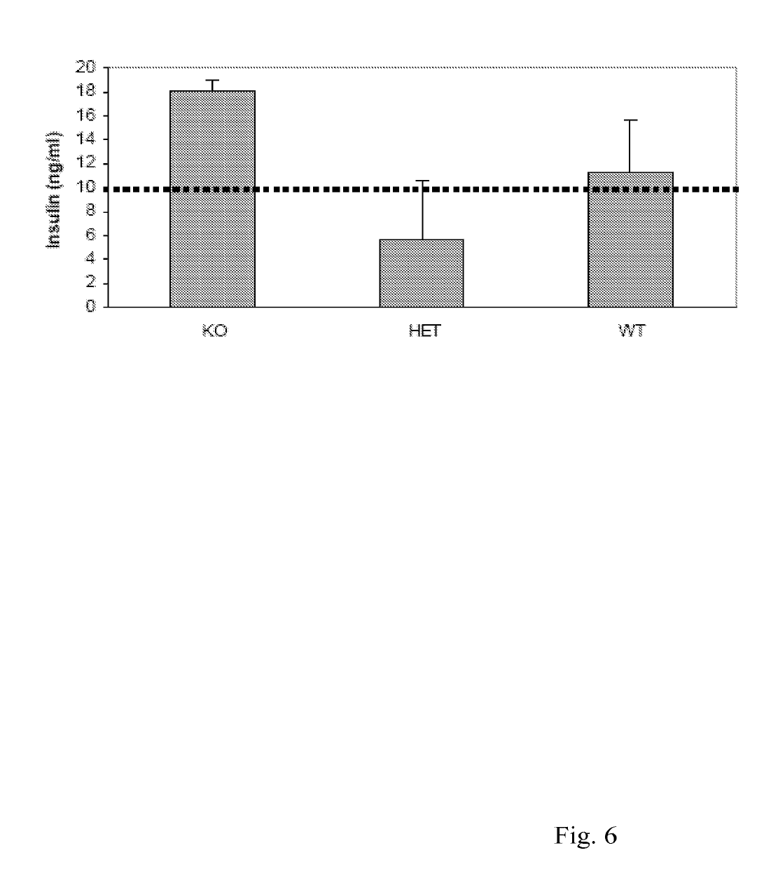

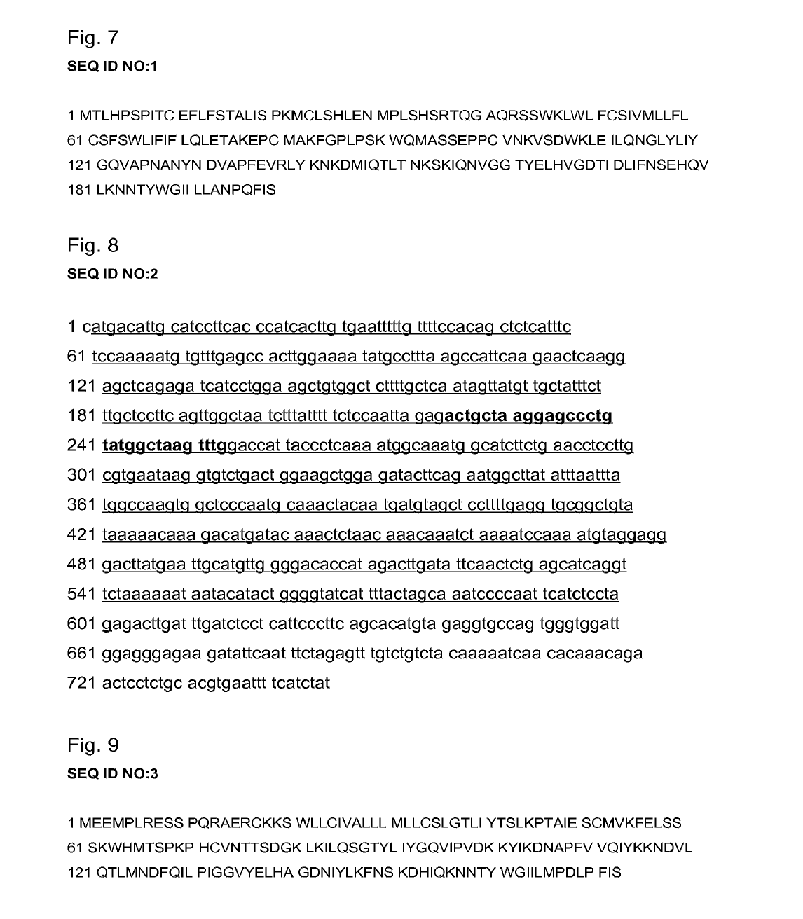

FIG. 6 shows serum insulin levels in 6 month old male mice. The dotted line represents the upper bound of normal serum insulin. Analysis of serum in the Gitrl−/− mice revealed elevated insulin compared to the wild type mice.

Preliminary clinical chemistry performed, on serum from 3 female, N9, Gitrl−/− mice (7 months old) has indicated that these mice had raised liver enzymes (ALT, AST), and increased blood cholesterol and glucose compared with age matched WT C57B1/6J controls as shown in the table below. These mice also had fatty change in their livers at this 9th generation.

TABLE
Serum biochemistry of female C57Bl/6J WT control and
9 generation backcrossed Gitrl−/− mice.

| | ALP | ALT | AST | Protein | Albumin | cholesterol | Glucose | Triglycerides | LDH |
|---|---|---|---|---|---|---|---|---|---|
| | U/l | U/l | U/l | g/l | g/l | mmol/l | mmol/l | mmol/l | U/l |
| control | 92 | 33.9 | 80.0 | 50.7 | 25.8 | 2.06 | 10.08 | 0.70 | 654 |
| control | 84 | 35.9 | 67.4 | 54.1 | 28.7 | 2.64 | 12.66 | 0.67 | 488 |
| GITRL— | 70 | 50.3 | 89.1 | 54.0 | 26.3 | 4.23 | 19.03 | 0.53 | 884 |
| GITRL— | 67 | 92.3 | 115.8 | 57.2 | 29.0 | 4.92 | 13.65 | 0.54 | 686 |
| GITRL— | 75 | 70.3 | 100.5 | 58.2 | 29.3 | 5.22 | 21.56 | 0.84 | 754 |

In summary, the knock-out of the GITRL gene confers on mice of the C57/B6 strain a propensity for weight gain (obesity), development of fatty liver, increased levels of ALT, AST, lactate dehydrogenase, cholesterol, glucose, insulin and interleukin-6 in the blood. This data argues for a hitherto unexpected role of GITR-ligand as a physiological regulator of metabolism. Specifically, the GITR-ligand acts on its receptor to normally prevent obesity and the development of metabolic syndrome and type 2 diabetes.

### Example 2

### Generation of an Inducible Gitrl−/− Knockout Mouse

We will generate inducible GITRL−/− mice directly in a C57BL/6 strain background. LoxP sites will be introduced flanking exon 1 to facilitate Cre-mediated conditional removal of the entire exon. This can be achieved by crossing with mice expressing the Cre-transgene either ubiquitously or in a tissue specific or inducible manner. A neomycin selection cassette inserted downstream of exon 1 during the introduction of the loxP sites will be flanked by FRT sites so that there is an option to delete it independently, prior to removal of exon 1, using FLPe recombinase. The upstream loxP site will be introduced into the 5′ untranslated region to avoid disruption of promoter elements. Deletion of exon 1 should result in the generation of a non-functional GITRL transcript lacking both the wild type initiation codon and the sequence encoding both the cytoplasmic and transmembrane domains.

### Example 3

### Further Analysis of Knockout Mouse Gitrl−/−

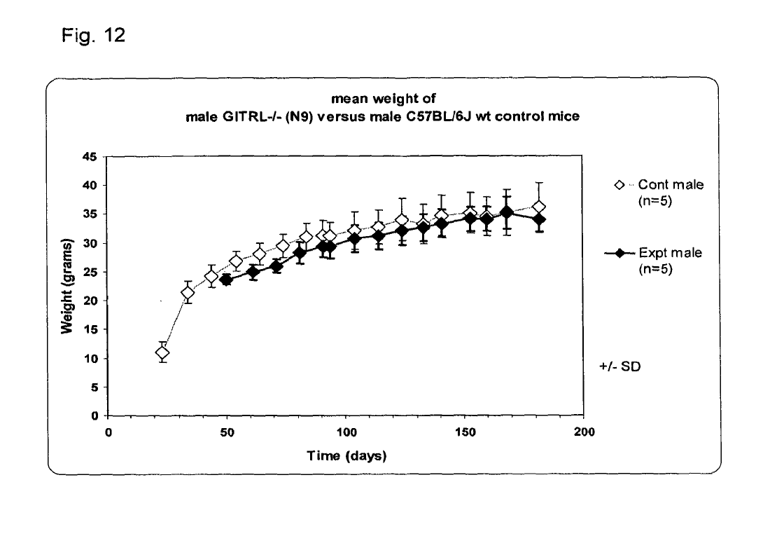

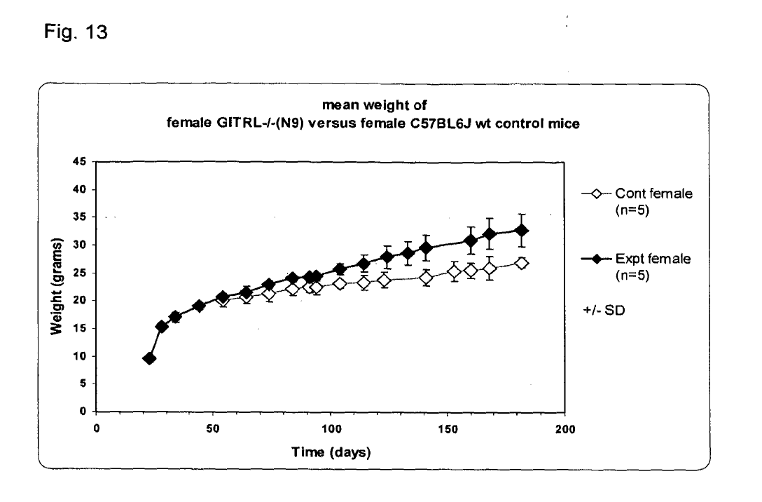

Groups of N9 GITRL−/− knockout mice as described in Example 1 were compared to groups of age and sex matched control wild type C57BL/6J. FIG. 12 shows the weight of male control and knockout mice over time. FIG. 13 shows the weight of female control and knockout mice over time. In both figures E denotes experimental (i.e. GITRL−/− knockout) mice and C denotes control (i.e. wild type C57BL/6J) mice.

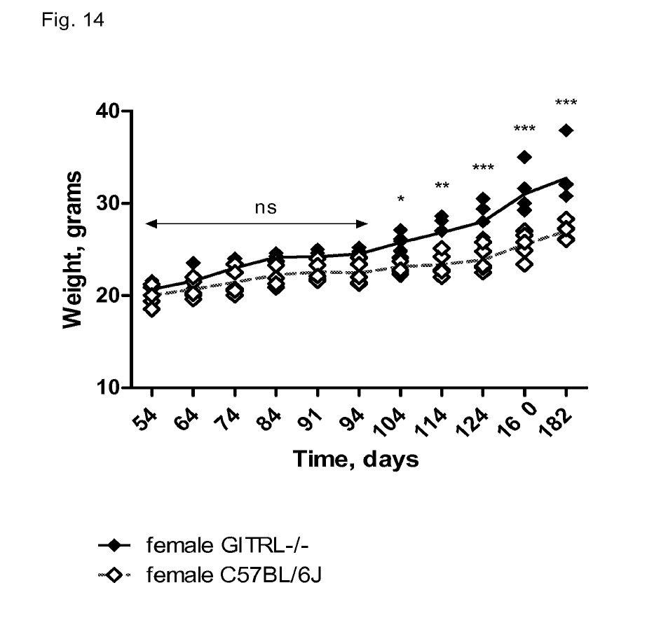

The mean weight of the female experimental mice began to diverge from that of the controls by about 10 weeks of age and by the final time point of 26 weeks experimental female mice were on average approximately 20% heavier than controls. FIG. 14 shows that the weight differential is statistically significant from ˜100 days. 2-way ANOVA, with Bonferroni post test, was used to determine if the weight difference is significant at a given time point. P value: ***<0.001; **0.001-0.01; *0.01-0.05; ns>0.05. The weight differential is statistically significant with p value 0.01-0.05 at 104 days, with p value 0.001-0.01 at 114 days, and with p value <0.001 at 124 days, 160 days and 182 days.

In contrast, the mean weight of the male experimental mice was just below that of the controls for the first 90-100 days and equivalent at 26 weeks. They were only about 4% heavier than the age matched experimental females.

The propensity for weight gain seen in GITRL−/− knockout mice is therefore gender dependant. However, weight gain can be considered as an end result. Although the male knockout mice showed little, if any, weight gain when compared to the control mice, other metabolic changes were observed in these mice as set out in Example 1 above and the examples below. It is likely that there are compensatory mechanisms coming into play that may obscure the development of obesity, which then overlaps with “natural” obesity in these caged mice.

As noted in Example 1, FIGS. 3 and 4 provide clear evidence of fat deposition in the livers of female experimental mice at 6 months of age. Further analysis of H&E stained livers suggests that this deposition is not yet apparent by 16 weeks. Livers have been archived from experimental and control female mice at 8, 12, 16 and 26 weeks and from male mice at 26 weeks. The development of fatty change will confirm that removal of GITRL, or alternatively interfering with its ability to engage its receptor, does give a disease defined both by clear pathological indicators and metabolic parameters in both genders.

### Example 4

### Clinical Analysis

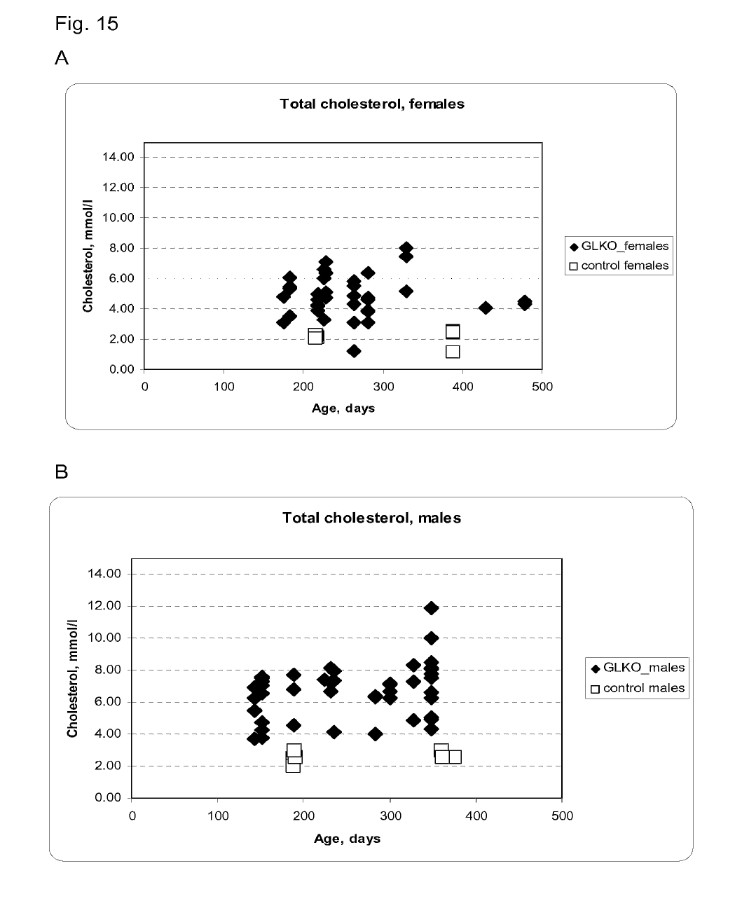

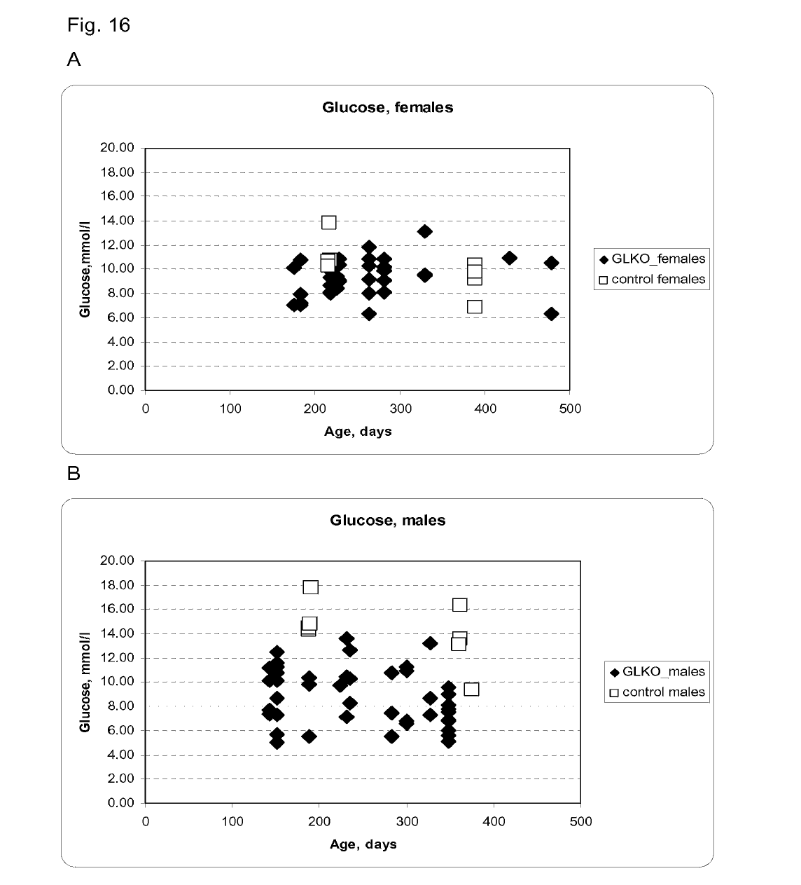

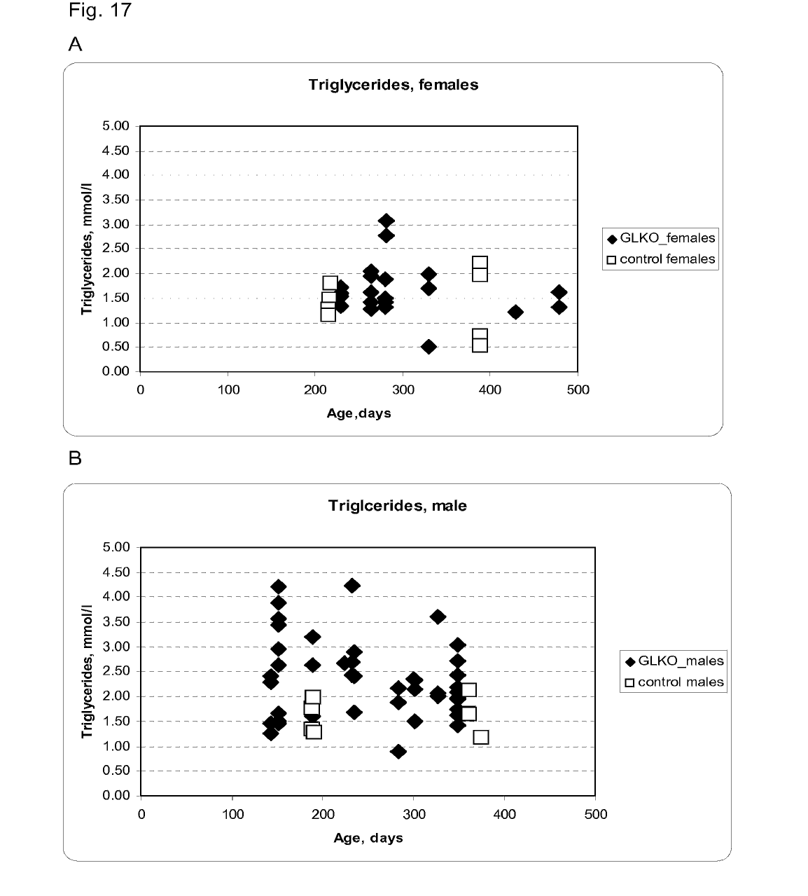

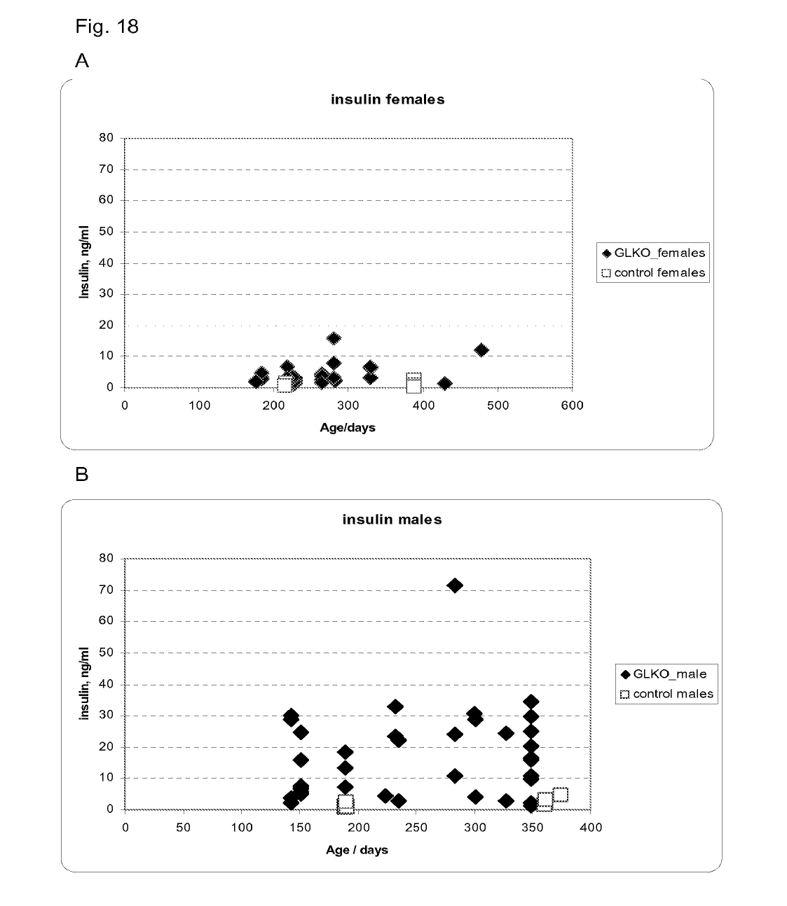

Terminal weights and serum for clinical analysis were obtained from 36 female (age 25-68 weeks) and 45 male (age 20-50 weeks) GITRL−/− mice (N9). Both genders exhibited a similar and consistent increase in cholesterol relative to C57BL/6J control mice across the age range analysed (FIG. 15). Glucose levels were not raised in the female mice, but were decreased in male mice relative to controls (FIG. 16). The level of triglycerides did not vary with time in knockout and control mice over a period >150 days to >1 yr. While it was similar in the female knockout and the female control mice it was elevated in the male knockout mice compared with male control mice (FIG. 17). Higher levels of insulin were observed in the knockout mice from age >150 days to >1 yr as compared to the control mice, and this effect was more pronounced in male mice compared with female mice (FIG. 18). Over this time period, the levels of insulin remained at a constant level in the knockout mice and in the wild type mice. These data highlighted a clear impact of gender on the metabolic effect of GITRL insufficiency.

### Example 5

### Metabolomic Analysis

The goal of this study was to characterize biochemical changes in plasma from mice lacking TNFSF18 ligand (GITRL) compared to wild type animals at 8, 12, and 16 weeks. Plasma was collected from CO₂-anesthetized female wild type (WT, C57BL/6J) and GITRL homozygous mutant, (GLKO, backcrossed to C57BL6, N9), mice at 8 weeks of age (w), 12 w, and 16 w, 5 animals per group.

| | | |
|---|---|---|
| | No. of | |
| Group | samples | Description |

| | | | |
|---|---|---|---|
| WT | 8 w | 5 | plasma from 8 week old wild type, |
| | | | C57BL/6J female mice |
| GLKO | 8 w | 5 | plasma from 8 week old |
| | | | GITRL −/− female mice |
| WT | 12 w | 5 | plasma from 12 week old wild type, |
| | | | C57BL/6J female mice |
| GLKO | 8 w | 5 | plasma from 12 week old |
| | | | GITRL −/− female mice |
| WT | 16 w | 5 | plasma from 16 week old wild type, |
| | | | C57BL/6J female mice |
| GLKO | 8 w | 5 | plasma from 16 week old |
| | | | GITRL −/− female mice |

For metabolomic analysis, samples were extracted and prepared using Metabolon's standard solvent extraction method. The extracted samples were split into equal parts for analysis on the GC/MS and LC/MS/MS platforms. Also included were several technical replicate samples created from a homogeneous pool containing a small amount of all study samples.

The table below shows the number of gene changes between the knockout and wildtype mice at each time point. Welch's Two Sample t-tests were used to determine whether the means of two populations are different.

| | GLKO | | |
|---|---|---|---|
| | WT | | |
| Welch's Two Sample t-Test | 8 w | 12 w | 16 w |
| Total number of biochemicals with | 72 | 60 | 11 |
| p ≦ 0.05 | | | |
| Biochemicals (↑↓) | ↑40-↓32 | ↑18-↓42 | ↑5-↓6 |
| q-value (for p ≦ 0.05) | 0.07 | 0.10 | 0.87 |

The global metabolic profiles of GITRL−/− knockout versus wild type plasma samples were profoundly different at the earliest observed time-point, 8 weeks of age, suggesting that genotype dependant differences in metabolism are well established in young adult mice. Clear differences in plasma metabolic profiles between GITRL−/− and wildtype mice were observed across a time course of 8 to 16 weeks, including changes in arginine metabolism, amino acid metabolism and energy substrate selection. A large number of compounds showed a significant age by genotype effect. By the 16 week time point there were fewer differences, suggesting normalisation over time due to the effect of stabilizing compensatory metabolism, or, considering that many metabolites displayed opposing trends by genotype it may be that the 16 week time point represents a cross-over point.

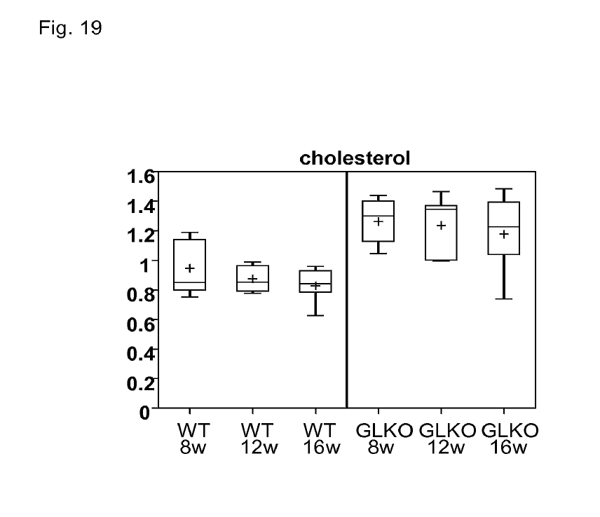

FIGS. 19 to 22 show results from the metabolomic analysis.

FIG. 19 shows the metabolomic data obtained for cholesterol in control and knockout mice over time. GLKO denotes GITRL−/− knockout mice and WT denotes control (i.e. wild type C57BL/6J) mice. Higher levels of cholesterol were observed in the knockout mice at 8 weeks as compared to the control mice. Over the time course, the levels of cholesterol remained at a constant high level in the knockout mice, whilst they remained at a constant lower level in the wild type mice. The data in Example 4 and FIG. 15 confirms that this differential was retained to >1 yr of age.

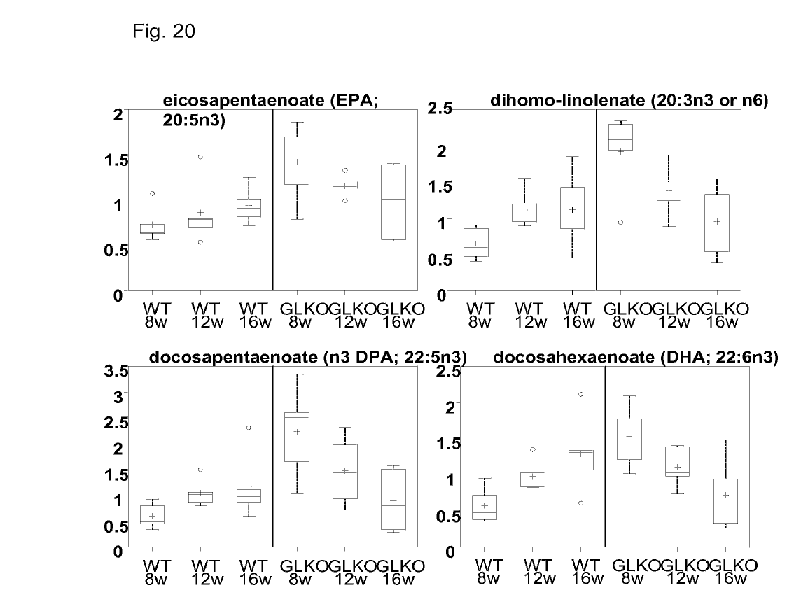

FIG. 20 shows the metabolomic data obtained for essential fatty acids in control and knockout mice over time. GLKO denotes GITRL−/− knockout mice and WT denotes control (i.e. wild type C57BL/6J) mice. Higher levels of these free fatty acids (FFAs) (i.e. eicosapentaenoate (EPA; 20:5n3), docosapentaenoate (n3 DPA; 22:5n3), dihomo-linolenate (20:3n3 or n6) and docosahexaenoate (DHA; 22:6n3)) were observed in the knockout mice at 8 weeks as compared to the control mice. In general the wildtype mice showed slightly increasing levels of FFAs over the time course from 8 weeks to 16 weeks. In contrast the knockout mice showed relatively high levels of these fatty acids at 8 weeks and these levels decreased over the time course from 8 weeks to 16 weeks.

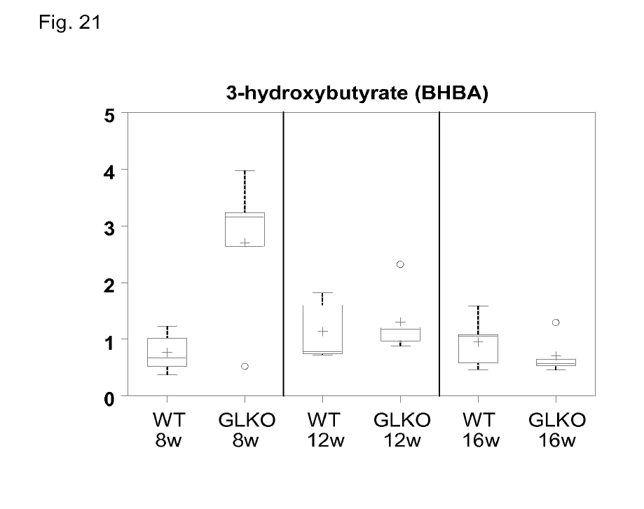

FIG. 21 shows the metabolomic data obtained for 3-hydroxybutyrate (BHBA) in control and knockout mice over time. GLKO denotes GITRL−/− knockout mice and WT denotes control (i.e. wild type C57BL/6J) mice. At the 8 week timepoint, the knockout mice showed much higher levels of BHBA than the wildtype mice. Over the time course, the levels of BHBA decreased in the knockout mice whilst they remained unchanged in the wild type mice.

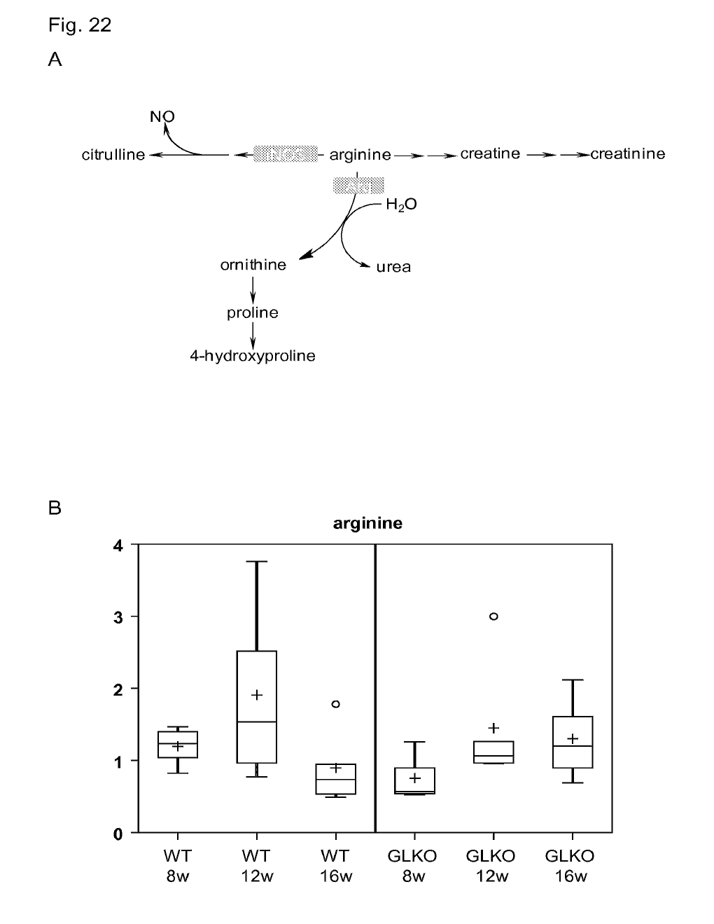

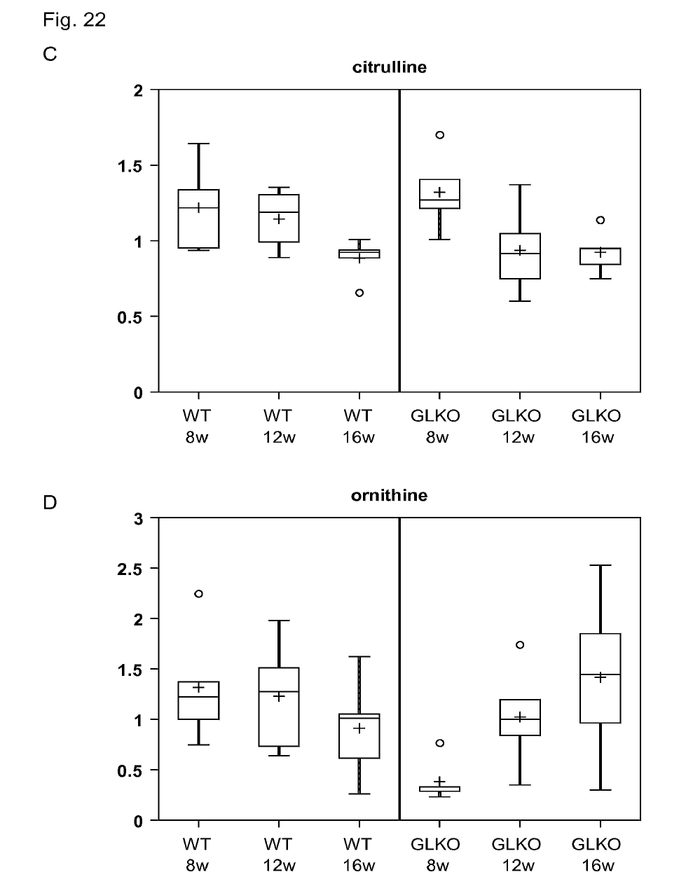

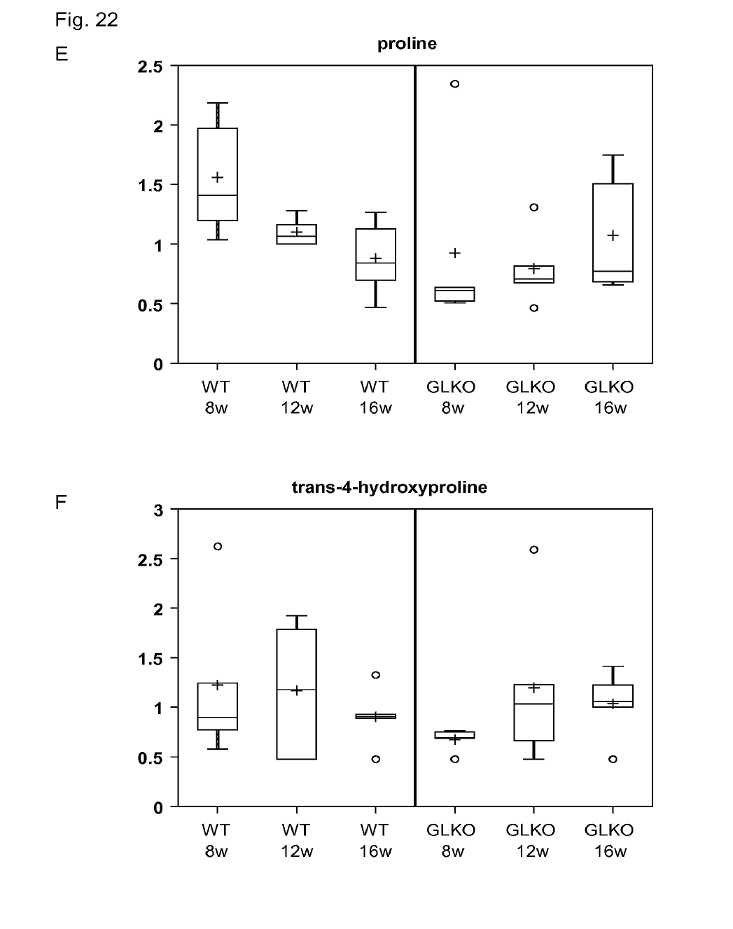

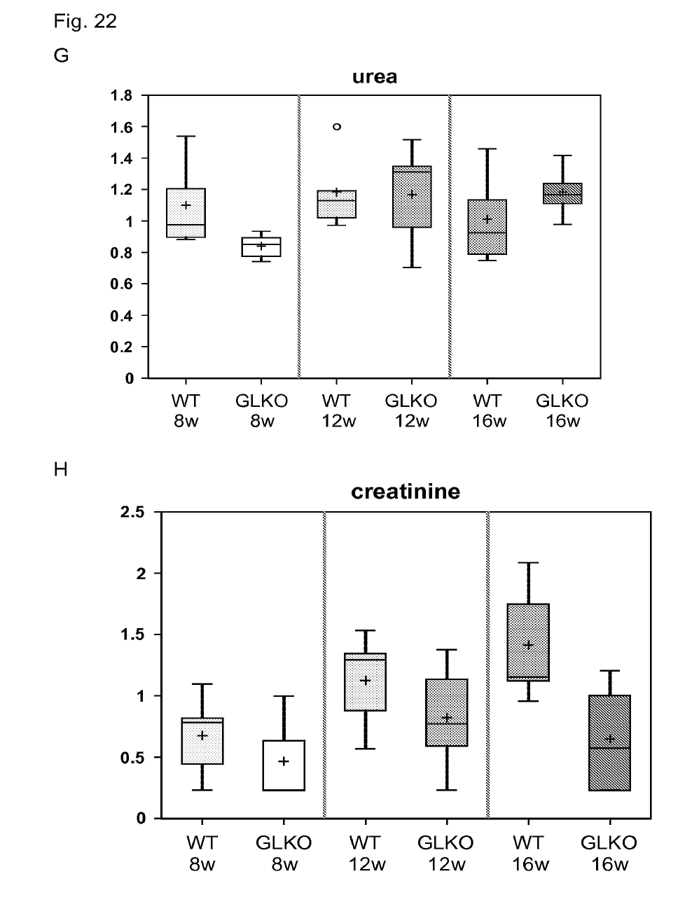

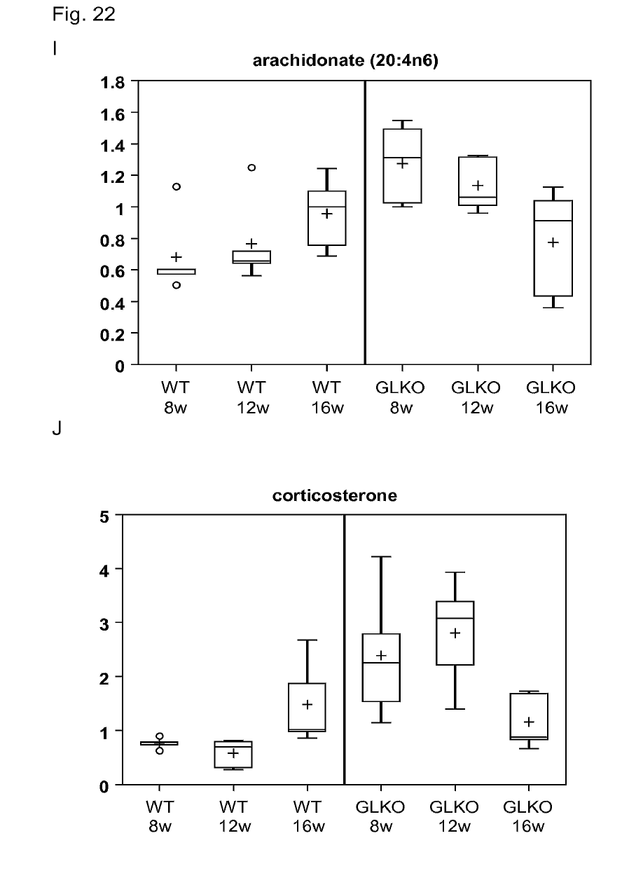

FIG. 22 shows the different metabolic pathways for breaking down arginine (A) and metabolomic data obtained for arginine (B), citrulline (C), ornithine (D), proline (E), trans-4-hydroxyproline (F), urea (G), creatine (H), arachidonate (I) and corticosterone (J) in control and knockout mice over time. GLKO denotes GITRL−/− knockout mice and WT denotes control (i.e. wild type C57BL/6J) mice.

Arginine can be metabolized along three distinct pathways reflecting the relative activities of the enzymes iNOS and arginase (Arg), as well as the creatine pathway. Initially, arginine metabolism in the GITRL−/− knockout mice appeared to be biased towards citrulline (and thus NO production) and creatinine metabolites, indicating relatively decreased activity along the Arg pathway compared to WT. By the 16w time point, the relative activity of the arginine metabolic pathways showed reversed emphasis with elevated levels of ornithine and ornithine catabolites, but relatively lower levels of the citrulline/NO and creatine pathway metabolites. These compound level changes are indicative of variations in the iNOS pathways of immune response and inflammation.

Lower levels of arginine were observed in the knockout mice at 8 weeks as compared to the control mice (FIG. 22B). Over the time course, the levels of arginine increased in the knockout mice whilst they decreased slightly in the wild type mice.

Higher levels of citrulline were observed in the knockout mice at 8 weeks as compared to the control mice (FIG. 22C). Over the time course, the levels of citrulline decreased in the knockout mice whilst they decreased less in the wild type mice.

Lower levels of ornithine were observed in the knockout mice at 8 weeks as compared to the control mice (FIG. 22D). Over the time course, the levels of ornithine increased in the knockout mice whilst they decreased slightly in the wild type mice.

Lower levels of proline were observed in the knockout mice at 8 weeks as compared to the control mice (FIG. 22E). Over the time course, the levels of proline increased in the knockout mice whilst they decreased in the wild type mice.

Lower levels of trans-4-hydroxyproline were observed in the knockout mice at 8 weeks as compared to the control mice (FIG. 22F). Over the time course, the levels of trans-4-hydroxyproline increased in the knockout mice whilst they decreased slightly in the wild type mice.

Over the time course, the levels of urea remained relatively constant in the wild type mice, whilst the levels increased slightly in the knockout mice (FIG. 22G).

Higher levels of creatine were observed in the control mice at 16 weeks as compared to the knockout mice (FIG. 22H). Over the time course, the levels of creatine increased in the wild type mice whilst they increased initially from 8 weeks to 12 weeks in the knockout mice, but then decreased from 12 weeks to 16 weeks.

Higher levels of arachidonate were observed in the knockout mice at 8 weeks as compared to the control mice (FIG. 221). Over the time course, the levels of arachidonate decreased in the knockout mice whilst they increased in the wild type mice.

Higher levels of corticosterone were observed in the knockout mice at 8 weeks as compared to the control mice (FIG. 22J). Over the time course, the levels of corticosterone decreased in the knockout mice whilst they increased slightly in the wild type mice.

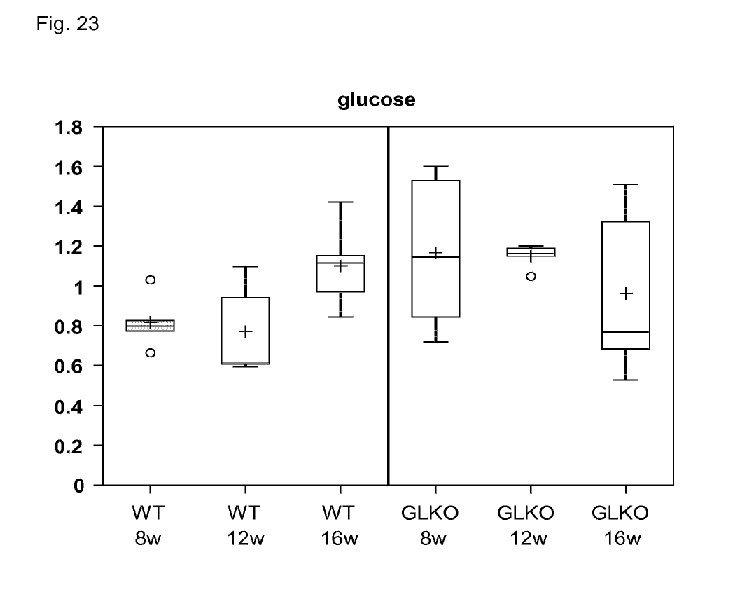

FIG. 23 shows the metabolomic data obtained for glucose in control and knockout mice over time. GLKO denotes GITRL−/− knockout mice and WT denotes control (i.e. wild type C57BL/6J) mice. Higher levels of glucose were observed in female knockout mice at 8 weeks as compared to female control mice. Over the time course, the elevated level of glucose levelled from 12 to 16 weeks in the knockout mice, whilst equivalently elevated levels were also detected in the wild type mice at 16 weeks. The clinical data shown in FIG. 16a confirms that equivalent levels of glucose were maintained in female knockout and control mice to >1 yr. Glucose levels in male knockout mice at equivalent later time points were decreased compared with control male mice.

This data provides a set of metabolic parameters which are indicative of a disease process—not only implicating a role for GITRL, but also providing a set of biomarkers that will allow the disease prediction, diagnosis and the study of therapeutic intervention.

Sustained elevated cholesterol levels were validated from 8 weeks, as were early raised glucose levels (8 and 16 week). Changes in arginine metabolism, along with significant alterations in corticosterone and arachidonate, suggested an augmented inflammatory status in the GITRL−/− mice up to 12 weeks that is substantially resolved by the 16 week time-point. Genotype specific changes in amino acid metabolism were noted over time, as was an alteration in energy substrate selection. Early time-points showed significantly elevated levels of free fatty acids (FFAs), glucose and the ketone body BHBA in GITRL−/− plasma. Cellular substrate selection of glucose or lipids for energy metabolism can impact compound levels of FFAs, ketone bodies, and carbohydrates in plasma. Plasma glucose and lipid levels are modulated by tissue responses to insulin signaling and diet with significant contributions from adipose tissue lipolysis, liver gluconeogenesis /glycogenolysis, as well as tissue glucose and lipid uptake. Plasma FFAs reflect the combined impacts of cellular lipolysis, de novo fatty acid synthesis, and tissue uptake and degradation by fatty acid beta oxidation (FAO). Elevated plasma ketone body levels may occur when the level of acetyl-CoA from the combined effects of glycolysis and FAO exceeds the capacity of the TCA cycle and is instead used for ketogenesis.

Elevated plasma glucose, ketone bodies, and FFAs are reminiscent of metabolic signatures associated with insulin resistance/diabetes and metabolic perturbations frequently incurred with obesity. In this case, however, because elevated glucose, BHBA, and FFA in GITRL−/− plasma occurs at the early time points prior to the large increases in animal weights, it is unlikely that the differences are secondary results of obesity but rather may indicate a more direct impact of GITRL function on metabolism.

These metabolic studies reveal that there are metabolome changes that are demonstrable early in the GITRL−/− mice. Some of these are always raised above normal (e.g, cholesterol, insulin, glucose, IL6) reflecting disease associated metabolic and inflammatory events, and these are be considered as good primary endpoints for test treatments.

There are other metabolome changes that can be considered as secondary endpoints. These are more complex, and some may reflect more distal events of a pathway (e.g as if part of a signaling network), or compensatory events to maintain metabolic homeostasis and control of inflammation (e.g. changes in citrulline, arginine, proline, free fatty acids and the elevation of corticosterone and arachnidonate which decline as animals get older). Although there are likely to be rebound “control” responses, it is expected that changes will be seen in an individual over time. Therefore these markers will be valuable as part of a network of interacting parameters which could be usefully monitored (as in a systems biology approach).

1. A method for the prevention or treatment of a condition selected from type 2 diabetes, obesity and metabolic syndrome, the method comprising administering to a subject in need thereof, a GITR-ligand or an analogue thereof or an agonist of a GITRL-associated receptor, preferably a GITR-binding molecule or an antigen-binding fragment thereof.

2. A method for lowering cholesterol in a mammal said method comprising administering to said mammal a GITR-ligand or an analogue thereof or an agonist of a GITRL-associated receptor, preferably a GITR-binding molecule or an antigen-binding fragment thereof.

3-4. (canceled)

5. A method according to claim 1, wherein the GITR-ligand analogue or the agonist of a GITRL-associated receptor or the GITR-binding molecule or antigen-binding fragment is an antibody or antibody fragment thereof.

6. A method according to claim 5 wherein the antibody or antibody fragment is a monoclonal antibody.

7. A method according to claim 5 wherein the antibody or antibody fragment is a chimeric antibody or fragment thereof and/or a humanized antibody or fragment thereof

8. A method according to claim 5, wherein the antibody or antibody fragment is an anti-GITR antibody or antibody fragment.

9. A method according to claim 1, wherein the GITR-ligand analogue or the agonist of a GITRL-associated receptor is a small drug analogue or agonist.

10. A method according to claim 1 further comprising administering one or more compounds selected from appetite suppressants, sulphonylureas, meglitinides, biguanides, thiazolidinediones, insulin, alpha glucosidase inhibitors, anti-hyperglycemic medications, DPP IV inhibitors and ACE inhibitors.

11-12. (canceled)

13. A pharmaceutical composition comprising a GITR-ligand or an analogue thereof or an agonist of a GITRL-associated receptor for use in the prevention or treatment of a condition selected from type 2 diabetes, obesity and metabolic syndrome or for use in lowering cholesterol in a mammal.

14. (canceled)

15. A method for screening for a substance, or a salt or a solvate thereof, to be used in the prevention or treatment of a condition selected from type 2 diabetes, obesity and metabolic syndrome, which comprises the following steps:
(i) measuring the severity of a symptom or sign of the condition in a non-human animal in which the GITRL gene is disrupted;
(ii) administering a test substance to said non-human animal;
(iii) measuring the severity of a symptom or sign of the condition of said non-human animal after administration of the test substance; and
(iv) comparing the severity of the symptom or sign of the condition before administration of the test substance with the severity of the symptom or sign of the condition after administration of the test substance, wherein a decrease or lessening of the symptom or sign indicates that the substance administered may be useful in the prevention or treatment of the condition.

16. A method according to claim 15 wherein the symptom or sign to be measured is one or more of weight gain, fatty liver, levels of ALT, AST, lactate dehydrogenase, cholesterol, glucose, insulin, free fatty acids (FFAs), ketone bodies (e.g. BHBA), or interleukin-6, triglycerides, essential fatty acids (e.g. eicosapentaenoate, docosapentaenoate, dihomo-linolenate and docosahexaenoate), arginine, proline, ornithine, trans-4-hydroxyproline, corticosterone or arachnidonate in the blood.

17. A method for diagnosing or predicting the risk of developing a condition selected from type 2 diabetes, obesity and metabolic syndrome the method comprising: measuring the concentration of one or more biomarkers in the blood of or in a plasma sample from a subject, the one or more biomarkers being selected from the group consisting of ALT, AST, lactate dehydrogenase, cholesterol, glucose, insulin, free fatty acids (FFAs), ketone bodies (e.g. BHBA), interleukin-6, triglycerides, essential fatty acids (e.g. eicosapentaenoate, docosapentaenoate, dihomo-linolenate and docosahexaenoate), arginine, proline, ornithine, trans-4-hydroxyproline, corticosterone, arachnidonate or citrulline, and (i) comparing the concentration in the sample from the subject to the concentration of the biomarker in a healthy subject, wherein a change in the level of the biomarker is indicative of one or more of the conditions, and/or (ii) comparing the concentration to the concentration of the biomarker in the same subject taken at a different time point, wherein a change in the level of the biomarker over time is indicative of one or more of the conditions.

18. A method for monitoring the effectiveness of a treatment of a condition selected from type 2 diabetes, obesity and metabolic syndrome comprising measuring the concentration of one or more biomarkers in the blood of or in a plasma sample from the subject, wherein the one or more biomarkers are selected from ALT, AST, lactate dehydrogenase, cholesterol, glucose, insulin, free fatty acids (FFAs), ketone bodies (e.g. BHBA), interleukin-6, triglycerides, essential fatty acids (e.g. eicosapentaenoate, docosapentaenoate, dihomo-linolenate and docosahexaenoate), arginine, proline, ornithine, trans-4-hydroxyproline, corticosterone, arachnidonate or citrulline.

19. A method for selecting an appropriate therapy for treating a condition selected from type 2 diabetes, obesity and metabolic syndrome in a patient comprising measuring the concentration of one or more biomarkers in the blood of or in a plasma sample from the subject, wherein the one or more biomarkers are selected from ALT, AST, lactate dehydrogenase, cholesterol, glucose, insulin, free fatty acids (FFAs), ketone bodies (e.g. BHBA), interleukin-6, triglycerides, essential fatty acids (e.g. eicosapentaenoate, docosapentaenoate, dihomo-linolenate and docosahexaenoate), arginine, proline, ornithine, trans-4-hydroxyproline, corticosterone, arachnidonate or citrulline.

20-25. (canceled)

26. A method according to claim 2, wherein the GITR-ligand analogue or the agonist of a GITRL-associated receptor or the GITR-binding molecule or antigen-binding fragment is an antibody or antibody fragment thereof.

27. A method according to claim 26, wherein the antibody or antibody fragment is an anti-GITR antibody or antibody fragment.

28. A method according to claim 26, wherein the antibody or antibody fragment is a monoclonal antibody.

29. A method according to claim 26, wherein the antibody or antibody fragment is a chimeric antibody or fragment thereof and/or a humanized antibody or fragment thereof.

30. A method according to claim 2 further comprising administering one or more compounds selected from appetite suppressants, sulphonylureas, meglitinides, biguanides, thiazolidinediones, insulin, alpha glucosidase inhibitors, anti-hyperglycemic medications, DPP IV inhibitors and ACE inhibitors.

31. A method according to claim 2, wherein the GITR-ligand analogue or the agonist of a GITRL-associated receptor is a small drug analogue or agonist.


SEQUENCE LISTING (12 sequences)

SEQ ID NO: 1 - protein, 199 aa, Homo sapiens
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 2 - DNA, 748 bp, Homo sapiens
catgacattgcatccttcacccatcacttgtgaatttttgttttccacagctctcatttctccaaaaatgtgtttgagccacttggaaaatatgcctttaagccattcaagaactcaaggagctcagagatcatcctggaagctgtggctcttttgctcaatagttatgttgctatttctttgctccttcagttggctaatctttatttttctccaattagagactgctaaggagccctgtatggctaagtttggaccattaccctcaaaatggcaaatggcatcttctgaacctccttgcgtgaataaggtgtctgactggaagctggagatacttcagaatggcttatatttaatttatggccaagtggctcccaatgcaaactacaatgatgtagctccttttgaggtgcggctgtataaaaacaaagacatgatacaaactctaacaaacaaatctaaaatccaaaatgtaggagggacttatgaattgcatgttggggacaccatagacttgatattcaactctgagcatcaggttctaaaaaataatacatactggggtatcattttactagcaaatccccaattcatctcctagagacttgatttgatctcctcattcccttcagcacatgtagaggtgccagtgggtggattggagggagaagatattcaatttctagagtttgtctgtctacaaaaatcaacacaaacagaactcctctgcacgtgaattttcatctat

SEQ ID NO: 3 - protein, 173 aa, Mus musculus
MEEMPLRESSPQRAERCKKSWLLCIVALLLMLLCSLGTLIYTSLKPTAIESCMVKFELSSSKWHMTSPKPHCVNTTSDGKLKILQSGTYLIYGQVIPVDKKYIKDNAPFVVQIYKKNDVLQTLMNDFQILPIGGVYELHAGDNIYLKFNSKDHIQKNNTYWGIILMPDLPFIS

SEQ ID NO: 4 - DNA, 2064 bp, Mus musculus
ttgtgggtatctgctttccccagttctcattccatcagagaacgagttctagcctcatggaggaaatgcctttgagagaatcaagtcctcaaagggcagagaggtgcaagaagtcatggctcttgtgcatagtggctctgttactgatgttgctctgttctttgggtacactgatctatacttcactcaagccaactgccatcgagtcctgcatggttaagtttgaactatcatcctcaaaatggcacatgacatctcccaaacctcactgtgtgaatacgacatctgatgggaagctgaagatactgcagagtggcacatatttaatctacggccaagtgattcctgtggataagaaatacataaaagacaatgcccccttcgtagtacagatatataaaaagaatgatgtcctacaaactctaatgaatgattttcaaatcttgcctataggaggggtttatgaactgcatgctggagataacatatatctgaagttcaactctaaagaccatattcagaaaactaacacatactgggggatcatcttaatgcctgatctaccattcatctcttagagattgggtttggtctcctcatcttcttctttgtatcccgagatgctggtgggtgggttggagggggatgattgatggcaatgcacacagtttgtgagggcttacaaattgacacaatcagagcctcttggcatataaaattttagccctcatatctgtctgaagaggactcagcaaatgggccaatccctaatgttgggtctgcaaatggacttgtacaatccatgataaaaaggagtatgggccacagaagacagaaactcttccaaagaatgtctttctaaccttgatccctgggtagaatgagatcctgtttccatgggagtcttacttggcttgcaaaaaagggtgtagggcagtagcttggccttttttccatcataatttccttgagctgttttaccttaatccctccaaactctcaccttctgagagcctcctaatgaaacattgttagactggtggggtggccaagacatgccaacaacacccttctttagaggtggtgtttttagaggacagagaacattatgaagcctagagcagcagaggtcaagatgccacgaaatggaattgatctgggaattttttttttttttcattctcaggatgcaggttcattctgaactttcccctaggccttcattgcttttgtgtgtatgtgtgcataaattctgcaaatagaaaaatgagagtttgcaccagtactcactagatttaacaccagaaagtggtacttttctggctgtattatgccatgatagcacattttctgttggtgttccctaactgacaagtataacagttttcctaaaccacacaacaatgctatgatgttaatggggtagatatttttggaaaaaaattgcacagtgagaacatgggtagatgaaccctaagactcttacctcaattcagaactcgcaaggagttaagtgagtggggtcttcattagaccattcacatggtctctgctttgaaactggcgttgctactgtctcattatacatcactaaaatggaattaactcaactttgaaatggatgcatcgactttaccccaaggtgtccagaatgaagctacaagacttttaccagcagtcattttccttttgcctggagcaagaagatccaggatactgttggaagagttcatctcactcaaccatgctgactttccaaagtaataatgaacatttgtgttcaaattttggattctgttaaatttagccagcttgtgagttcttgtcgaaaagtattttaaaccaatttacactatttatgggtatttgtgaaaagctatatagtgatattttatatataactaatttaaaatatttttattttatgtaacaaaaatactataggctaagctatttcttcttatttttttatgaatacttgctgaattgccatagggcacaaagactcttctgtttgcatatcttctcaggaaattaaaattgtatcacatgtatttataagaa

SEQ ID NO: 5 - DNA, 1261 bp, Mus musculus
acttgtgggtatctgctttccccagttctcattccatcagagaacgagttctagcctcatggaggaaatgcctttgagagagtcaagtcctcaaagggcagagaggtgcaagaagtcatggctcttgtgcatagtggctctgttactgatgctgctctgttctttgggtacactgatctatacttcactcaagccaactgccatcgagtcctgcatggttaagtttgaactatcatcctcaaaatggcacatgacatctcccaaacctcactgtgtgaatacgacatctgatgggaagctgaagatactgcagagtggcacatatttaatctacggccaagtgattcctgtggataagaaatacataaaagacaatgcccccttcgtagtacagatatataaaaagaatgatgtcctacaaactctaatgaatgattttcaaatcttgcctataggaggggtttatgaactgcatgctggagataacatatatctgaagttcaactctaaagaccatattcagaaaaataacacatactgggggatcatcttaatgcctgatctaccattcatctcttagagattgggtttggtctcctcatcttcttctttgtatcccaccctctctgtgtatgtgttatgttgtttgtatgtattgcatgcaattatttaggtgtgcacatgtatgtgttgtgcgagagcctgtgcaagtgtgtatgtgcttgtgggtgtggcactgagtgtcttccttagttcctctaccccttaatctttgagacgtggtgatctctccctgaacccagagttcatctatttggcttggcctgcccaccatttggctctaaggatctagctattgctgctcaccagccacagttctcagattacaggtgtgtgccgctatgcctggatatcacatggattttggagatctaaacttaggtccttatacttacatagcaggcatgctatccacagagttatttctggaacttaatgatgtcattctgtccctgtcttattcttaggacaatcaaggcagatagaaaacgaaaaaaatgatattgccacatacccagcaaaggcccaacagaattgtgatttttttttaagtaccagtgggattcagacagagggatagctagcaacatatagcatctgaatttcttccaatgttatcatttcagcttttgcgttcaatgtatactcaactctttttctctattttaaaactacaataaaatatgtatttataattgta

SEQ ID NO: 6 - protein, 12 aa, Artificial
  Synthetic peptide
GFSLSTSGMGVG

SEQ ID NO: 7 - protein, 16 aa, Artificial
  Synthetic peptide
HIWWDDDKYYNPSLKS

SEQ ID NO: 8 - protein, 16 aa, Artificial
  Synthetic peptide
HIWWDDDKYYQPSLKS

SEQ ID NO: 9 - protein, 9 aa, Artificial
  Synthetic peptide
TRRYFPFAY

SEQ ID NO: 10 - protein, 11 aa, Artificial
  Synthetic peptide
KASQNVGTNVA

SEQ ID NO: 11 - protein, 7 aa, Artificial
  Synthetic peptide
SASYRYS

SEQ ID NO: 12 - protein, 9 aa, Artificial
  Synthetic peptide
QQYNTDPLT